(19) United States
(12) Patent Application Publication
(10) Pub. No.: US 2011/0097279 A1
(43) Pub. Date: Apr. 28, 2011

(54) STABLE NON-ALCOHOLIC FOAMABLE PHARMACEUTICAL EMULSION COMPOSITIONS WITH AN UNCTUOUS EMOLLIENT AND THEIR USES

(75) Inventors: Dov TAMARKIN, Ness Ziona (IL); Meir EINI, Ness Ziona (IL); Doron FRIEDMAN, Karmei Yosef (IL); David SCHUZ, Moshav Gimzu (IL); Tal BERMAN, Rishon Lezion (IL)

(73) Assignee: FOAMIX LTD., Ness Ziona (IL)

(21) Appl. No.: 12/974,857

(22) Filed: Dec. 21, 2010

Related U.S. Application Data
(63) Continuation of application No. 11/940,290, filed on Nov. 14, 2007.
(60) Provisional application No. 60/858,747, filed on Nov. 14, 2006.
(60) Provisional application No. 60/899,176, filed on Feb. 2, 2007.

(51) Int. Cl. A61K 9/12; A61P 17/00; A61K 8/06; A61Q 19/00

(52) U.S. Cl. 424/45

(57) ABSTRACT

A stable non-alcoholic foamable pharmaceutical emulsion composition includes an unctuous emollient, at a concentration of about 0.5% to about 49% by weight; at least one multi-active agent; at a concentration of about 0.5% to about 15% by weight; water; an effective amount of an active pharmaceutical agent having a degree of solubility in the emulsion composition; and at least one liquefied or compressed gas propellant at a concentration of about 3% to about 25% by weight of the total composition; wherein the unctuous emollient comprises a petrolatum alone or in combination with other unctuous agents; wherein the multi active agent is selected from the group consisting of (a) two or more complex emulgators wherein there is a difference of about 4 or more units between the HLB values of two of the emulgators or there is a significant difference in the chemical nature or structure of two of the emulgators; (b) a surfactant and a foam adjuvant or co surfactant, wherein the surfactant has a HLB close to the required HLB of the oil phase; (c) a surfactant and a liquid wax, wherein the surfactant has a HLB close to the required HLB of the oil phase; (d) a surfactant and a polymeric agent other than starch or a modified starch ester, wherein the surfactant has a HLB close to the required HLB of the oil phase; (e) a polymeric agent and a foam adjuvant or co surfactant, which can cooperate to stabilize the emulsion; (f) a single surfactant without a long polymeric side chain that is composed of a mixture of esters having a HLB close to the required HLB of the oil phase; combinations of any of the above, and wherein the composition is substantially flowable is stored in an pressurized container and upon release expands to form a breakable foam.

### RELATED APPLICATIONS

This application claims the benefit of priority under 35 U.S.C. 119(e) to U.S. Application No. 60/858,747, filed Nov. 14, 2006, and entitled “Stable Non-Alcoholic Foamable Pharmaceutical Emulsion Compositions With An Unctuous Emollient And Their Uses,” which is hereby incorporated in its entirety by reference.

This application claims the benefit of priority under 35 U.S.C. 119(e) to U.S. Application No. 60/899,176, filed Feb. 2, 2007, and entitled “Non-Alcoholic Foamable Petrolatum-Based Pharmaceutical and Cosmetic Compositions And Their Uses,” which is hereby incorporated in its entirety by reference.

This application is a continuation under 35 U.S.C. 120 of co-pending U.S. application Ser. No. 11/940,290, filed Nov. 14, 2007, and entitled “Stable Non-Alcoholic Foamable Pharmaceutical Emulsion Compositions with an Unctuous Emollient and Their Uses,” which is hereby incorporated in its entirety by reference.

### BACKGROUND

This invention relates to unctuous emollient foamable pharmaceutical and cosmetic compositions.

Foams and, in particular, foam emulsions are complicated systems which do not form under all circumstances. Changes in foam emulsion composition, such as by the addition of active ingredients may destabilize the foam. There is, therefore, a need for a foam composition, which provides desirable properties to the skin and can remain stable whilst accommodating a variety of active ingredients.

Unctuous, e.g., having the characteristics of an oil or ointment, emollients have a number of useful attributes making them suitable candidates for topical foamable pharmaceutical and cosmetic compositions. They are inherently stable and inert which are clearly desirable characteristics. They are able to moisturize and soften the skin and in appropriate amounts can act as a protective or barrier layer and can form a barrier to water. By appropriate formulation they can act to improve drug delivery to the skin and yet remain resistant to being washed off. On the other hand they are by their nature greasy materials and can be difficult to formulate into a topical foamable composition that can deliver substantially uniform and stable foam that ameliorates or overcomes the look and feel of a greasy material. It is further a problem to incorporate into such a vehicle pharmaceutically effective amounts of one or more active pharmaceutical ingredients such that they are uniformly present throughout the formulation and are effectively delivered without the use of an alcohol in the formulation.

aliphatic alcohols in foam compositions promotes fast drying and thereby attempts to address the sticky feeling left by many topical formulations after application; however, alcohols, and in particular short chain alcohols like methyl, ethyl and isopropyl alcohols are defatting agents and may cause skin to become dry and cracked. Also, the presence of such alcohols generates alcoholic foam that is quick breaking, e.g., it collapses readily upon contact with a surface upon exposure to body temperature environment. Although certain compositions based on petrolatum are known they are, for example, designed to form an occlusive layer in the presence active pharmaceutical agents that are not soluble in the water or oil phase.

In the light of the unctuous, greasy, tacky, and heavy nature of petrolatum, there are problems in producing, stable emulsion foams of good quality and texture from high levels of petrolatum and there is a real technical challenge of achieving inter alia a good bubble structure, texture, spreadability and look and feel.

Alcohol is known to impair the integrity of the skin barrier, dry the skin and cause skin irritation. The incidence skin irritation (burning, itching and stinging) can be very high. Thus, while alcohol is useful in solubilizing an active agent and enabling effective dermal penetration of an active agent, the development of a safe foam vehicle, which will overcome the evident skin drying and irritation caused by alcohol, is warranted, especially where sensitive skin, mucosa, or body cavity membranes are being targeted.

Foamable compositions that produce foams, which are soft are desirable especially with improved stability.

It is particularly advantageous to have a foamable vehicle that is suitable for use as a base for delivery of not merely one type of API but is adaptable for use with one or more API's from a wide range of different types of API's with relatively minimal or minor adjustment to the vehicle. For example, by altering the amount of a component or by the addition of a buffer that provides a pH at which the API is stable as would be appreciated by a person skilled in the art.

### SUMMARY

Stable non-alcoholic foamable carriers and pharmaceutical emulsion compositions comprising an unctuous emollient, a multi-active agent, water, and a propellant with and without the addition of an active agent are described.

In another aspect, stable non-alcoholic foamable carriers and pharmaceutical emulsion compositions comprising an unctuous emollient, a multi-active agent, water, and a propellant with and without the addition of an active agent are described, wherein the foam produced by the carrier or pharmaceutical composition when packaged in an aerosol container and released has a foam hardness in the range of about 5 g to about 50 g. The greater the resistance of the foam to a force applied to it, and measured conveniently in grams, the greater the hardness of the foam. The reverse is that as the hardness is reduced the foams are softer. Foams having a foam hardness below about 50 g are comfortable for use. Foams with a hardness below about 40 g, preferably below about 35 g are soft foams; and below 20 g are very soft. In the light of the high viscosity of petrolatum compositions the foam produced is surprisingly soft especially given the high viscosity of the pre foam formulations.

Stable non-alcoholic foamable pharmaceutical emulsion compositions with an improved softness are described.

Stable non alcoholic foamable pharmaceutical emulsion compositions comprising an unctuous emollient, a multi-active agent, water, a propellant, and an active agent are described, wherein the composition, a phase of the composition or an unctuous component or an aqueous component of the emulsion is able to a degree to solubilize the active agent.

Stable non alcoholic foamable pharmaceutical emulsion compositions comprising an unctuous emollient, a multi-active agent, water, a propellant, and an active agent are described, wherein the unctuous and aqueous component is able to a very limited degree to solubilize the active agent and wherein the composition is formulated so that the resultant foam when applied topically to a target will not form an effective occlusive barrier, is not completely occlusive; or is not sufficient to form an occlusive barrier; or any occlusiveness is significantly transient; or so that the composition does not comprise an organic cosolvent.

Stable non alcoholic foamable pharmaceutical emulsion compositions comprising an unctuous emollient, a multi-active agent, water, a propellant, and an active agent are described, wherein the unctuous component and aqueous component are unable to a degree to solubilize the active agent and wherein the composition is formulated so that the resultant foam when applied topically to a target will not form an effective occlusive barrier, is not completely occlusive; or is not sufficient to form an occlusive barrier; or any occlusiveness is significantly transient; or so that the composition does not comprise an organic cosolvent.

A stable non-alcoholic foamable pharmaceutical emulsion composition includes an unctuous emollient, at a concentration of about 0.5% to about 49% by weight; at least one multi-active agent; at a concentration of about 0.5% to about 15% by weight; water; an effective amount of an active pharmaceutical agent having a degree of solubility in the emulsion composition; and at least one liquefied or compressed gas propellant at a concentration of about 3% to about 25% by weight of the total composition; wherein the unctuous emollient comprises a petrolatum alone or in combination with other unctuous agents; wherein the multi active agent is selected from the group consisting of (a) two or more complex emulgators wherein there is a difference of about 4 or more units between the HLB values of two of the emulgators or there is a significant difference in the chemical nature or structure of two of the emulgators; (b) a surfactant and a foam adjuvant or co surfactant, wherein the surfactant has a HLB close to the required HLB of the oil phase; (c) a surfactant and a liquid wax, wherein the surfactant has a HLB close to the required HLB of the oil phase; (d) a surfactant and a polymeric agent other than starch or a modified starch ester, wherein the surfactant has a HLB close to the required HLB of the oil phase; (e) a polymeric agent and a foam adjuvant or co surfactant, which can cooperate to stabilize the emulsion; (f) a single surfactant without a long polymeric side chain that is composed of a mixture of esters having a HLB close to the required HLB of the oil phase; combinations of any of the above, and wherein the composition is substantially flowable is stored in an pressurized container and upon release expands to form a breakable foam. By “close” as that term is used herein, it is meant to within about 3 HLB units, or preferably within about 2 HLB units or within about 1 HLB unit of the required HLB of the oil phase.

The present invention further relates to said composition comprising one or more additional active agents.

The present invention further relates to said composition comprising one or more additional therapeutically active oils.

In some embodiments, the foamable cosmetic or pharmaceutical composition is non-flammable, wherein said gas propellant contains hydrofluorocarbon.

The present invention further provides a method of treating, alleviating or preventing a disorder of mammalian subject, comprising administering a therapeutically effective amount of the above-mentioned compositions to an afflicted target site.

The present invention further provides use of a therapeutically effective amount of the above-mentioned compositions in the manufacture of a medicament.

The present invention further provides a therapeutically effective amount of the above-mentioned compositions for use in the manufacture of a medicament.

In one aspect, a stable non-alcoholic foamable emulsion composition comprises:
(1) an unctuous emollient consisting essentially of a petrolatum at a concentration of about 0.5% to about 60% by weight, and preferably 0.5% to about 49%;
(2) about 1% to about 49% liquid wax by weight,
(3) at least one multi-active agent; at a concentration of about 0.5% to about 15% by weight;
(4) water at a concentration of about 20% to about 50% of the formulation and
(5) at least one liquefied or compressed gas propellant at a concentration of about 3% to about 25% by weight of the total composition;
   wherein the composition is substantially flowable is stored in an pressurized container and upon release expands to form a breakable foam having a foam hardness in the range of about 5 g to about 50 g.

In some aspects, stable non-alcoholic foamable emulsion composition comprises:
(6) an unctuous emollient consisting essentially of a petrolatum at a concentration of about 0.5% to about 60% by weight, and preferably 0.5% to about 49%;
(7) about 1% to about 49% liquid oil by weight,
(8) at least one multi-active agent; at a concentration of about 0.5% to about 15% by weight;
(9) water at a concentration of about 20% to about 50% of the formulation and
(10) at least one liquefied or compressed gas propellant at a concentration of about 3% to about 25% by weight of the total composition;
   wherein the composition is substantially flowable is stored in an pressurized container and upon release expands to form a breakable foam having a foam hardness in the range of about 5 g to about 50 g.

### DETAILED DESCRIPTION

The present invention provides a safe and effective foamable pharmaceutical vehicle or composition. More particularly, it provides a stable non-alcoholic foamable pharmaceutical oil in water emulsion composition comprising an unctuous emollient and water. The vehicle or composition further comprises a multi-active agent.

In preparing stable non-alcoholic foamable pharmaceutical oil in water emulsion compositions containing an unctuous emollient suitable for delivery of an active pharmaceutical ingredient a combination of emulsifiers, a metal starch, and stabilizers were used to achieve a stable foam with petrolatum as the unctuous emollient.

In an embodiment of the present invention it was surprising found that it was possible to exclude stabilizer and still prepare a creamy stable foam without and with an active pharmaceutical ingredient.

In a still further embodiment it was surprisingly discovered that the replacement of the metal starch with a polymeric substance like a combination of carboxymethyl cellulose and microcrystalline cellulose or Arlacel 2121 and sucrose stearate was also effective in achieving a reasonably creamy stable foam without and with an active pharmaceutical ingredient.

It was also found that it was possible to eliminate the metal starch or polymeric agent and still achieve a reasonably creamy stable foam carrier. However, the introduction of clindomycin phosphate, as an example of a soluble active pharmaceutical ingredient, resulted in a significant loss in quality of the foam. So, it can be seen that the presence of a polymeric agent such as aluminium starch octenylsuccinate (“ASOS”), carboxymethyl cellulose sodium, and microcrystalline cellulose, or methocel and xanthan gum etc., can play a significant role and improve foam quality including, such as, hardness and stability.

In one or more embodiments of the present invention it is, further, possible to incorporate uniformly into an unctuous emollient foamable vehicle, pharmaceutically effective amounts of one or more active pharmaceutical ingredients.

In one or more embodiments of the present invention there is provided a foamable vehicle that is suitable for use as a base for delivery of not merely one type of API but is adaptable for use with one or more API's from a wide range of different types of API's with relatively minimal or minor adjustment to the vehicle. For example, by altering the amount of a component or by the addition of a buffer that provides a pH at which the API is stable as would be appreciated by a person skilled in the art.

In one or more embodiments of the present invention there is provided a foamable vehicle that is suitable for use as a base for delivery for API's, which are by their nature emulsion destabilizes, micelle destabilizers or interphase destabilizers, with relatively minimal or minor adjustment to the vehicle or in the method of preparation. Pharmaceutical salts, for example, can be in general, emulsion destabilizers. Anesthetics by virtue of their inherent function are likely to have an affinity for and may disturb the interphase. Pharmaceuticals that have a hydrophobic region and a hydrophilic region may sit across and affect the interphase. Thus, the identification of multi active agents that are effective in having anti destabilization properties in combination with an unctuous emollient of the present invention provides special advantages and is another embodiment of the present invention.

A stable non-alcoholic foamable pharmaceutical emulsion composition comprising:
an unctuous emollient, at a concentration of about 0.5% to about 49% by weight;
at least one multi-active agent; at a concentration of about 0.5% to about 15% by weight;
water;
an effective amount of an active pharmaceutical agent having a degree of solubility in the emulsion composition; and
at least one liquefied or compressed gas propellant at a concentration of about 3% to about 25% by weight of the total composition;
   wherein the unctuous emollient comprises a petrolatum alone or in combination with other unctuous agents;

wherein the multi active agent is selected from the group consisting of

(a) two or more complex emulgators wherein there is a difference of about 4 or more units between the HLB values of two of the emulgators or there is a significant difference in the chemical nature or structure of two of the emulgators;

(b) a surfactant and a foam adjuvant or co surfactant, wherein the surfactant has a HLB close to the required HLB of the oil phase;

(c) a surfactant and a liquid wax, wherein the surfactant has a HLB close to the required HLB of the oil phase;

(d) a surfactant and a polymeric agent other than starch or a modified starch ester, wherein the surfactant has a HLB close to the required HLB of the oil phase;

(e) a polymeric agent and a foam adjuvant or co surfactant, which can cooperate to stabilize the emulsion;

(f) a single surfactant without a long polymeric side chain that is composed of a mixture of esters having a HLB close to the required HLB of the oil phase;

combinations of any of the above;

and
wherein the composition is substantially flowable is stored in an pressurized container and upon release expands to form a breakable foam.

In a further embodiment of the present invention the foam hardness is in the range of about 8 g to about 40 g or more preferably 10 g to about 30 g.

In a further embodiment of the present invention the unctuous emollient influences foam hardness such that the foam produced is soft. Softness especially with stability improves usability.

In a further embodiment of the present invention the unctuous emollient is between about 3% to about 35% by weight of the composition.

In a further embodiment of the present invention the unctuous emollient is petrolatum, preferably between about 3% to about 35% by weight of the composition, more preferably between about 5% to about 30% by weight of the composition.

In a further embodiment of the present invention the multi active agent is preferably between about 1% to about 10% by weight of the composition.

In a further embodiment of the present invention the multi active agent and its amount is selected so that the composition is sufficiently shakable so that foam extrusion is not hampered. To this extent the maximum effective amount of multi active agent that may be used may be limited by the need for shakability.

In a further embodiment of the present invention the propellant is preferably between about 5% to about 12% by weight of the composition.

In an further embodiment of the present invention the degree of solubility of the active agent is slightly, sparingly or more soluble.

In an further embodiment of the present invention the degree of solubility of the active agent is very slightly soluble.

In a further embodiment of the present invention the active ingredient may be partially insoluble in one of the phases of the emulsion.

In a further embodiment of the present invention the active ingredient may be partially insoluble in the phases of the emulsion.

In a further embodiment of the present invention the active ingredient may be insoluble in one of the phases of the emulsion.

In a further embodiment of the present invention the active ingredient may be insoluble in the phases of the emulsion.

In one or more embodiments of the present invention the active ingredient may be insoluble or very slightly soluble in water or in the unctuous emollient, in which case one or more of the following can apply:
a) The composition is formulated so that the resultant foam when applied topically to a target will not per se form an effective occlusive barrier, is not completely occlusive; or is not sufficient to form an occlusive barrier or any occlusiveness is significantly transient; or
b) The composition does not comprise an organic cosolvent.

In a further embodiment of the present invention the active ingredient may be a cosmetic agent or a placebo. In which case, the carrier composition may itself be useful for the treatment prevention or amelioration of various general skin and cosmetic complaints such as aging, atopic dermititus, contact dermititus and radiation or burn injury and the like.

In one or more embodiments of the present invention the composition comprising one or more additional active agents.

In one or more embodiments of the present invention comprising one or more additional therapeutically active oils.

In some embodiments, the foamable cosmetic or pharmaceutical composition is non-flammable, wherein said gas propellant contains hydrofluorocarbon.

In one or more embodiments of the present invention there is provided a method of treating, alleviating or preventing a disorder of mammalian subject, comprising administering a therapeutically effective amount of the above-mentioned compositions to an afflicted target site.

In one or more embodiments of the present invention there is provided use of a therapeutically effective amount of the above-mentioned compositions in the manufacture of a medicament.

In one or more embodiments of the present invention there is further provided a therapeutically effective amount of the above-mentioned compositions for use in the manufacture of a medicament.

In one or more embodiments the unctuous emollient may alone or in combination with a multi active agent help to ameliorate, counteract, or overcome undesirable effects and drawbacks of an API, such as destabilization, on an emulsion vehicle, on a phase, on micelles or on an interphase.

In one or more embodiments the multi active agent may alone or in combination with an unctuous emollient help to ameliorate, counteract, or overcome undesirable effects and drawbacks of an API, such as destabilization, on an emulsion vehicle, on a phase, on micelles or on an interphase. Preferably the multi active agent comprises a polymeric agent such as ASOS, carboxymethyl cellulose sodium and microcrystalline cellulose or methocel and xantham gum.

Additionally, in one or more embodiments of the present invention there is provided foamable compositions that are stable and able to provide some of the main attributes of an unctuous emollient in a topical foamable formulation and which can deliver a substantially uniform and stable foam that ameliorates or overcomes the look and feel of a greasy material without the use of an alcohol in the formulation.

In one or more embodiments of the present invention there is provided a pharmaceutical foamable composition that can improve the solubility and/or deliverability of the active pharmaceutical to a target skin, mucosa or body cavity area.

In one or more embodiments a foamable pharmaceutical composition is provided also incorporating an added hydrophibic solvent, for example, as a look and feel enhancer, solubility enhancer or deliverability enhancer.

In one or more embodiments a foamable pharmaceutical composition is provided also incorporating an added polar solvent, for example, as penetration enhancer, solubility enhancer or deliverability enhancer.

In one or more embodiments, a pharmaceutical foamable composition is provided, wherein a pharmaceutical or a therapeutic active agent is incorporated in an unctuous emollient foamable vehicle, which contains additionally a hydrophobic solvent and a polar solvent.

In one or more embodiments a foamable pharmaceutical composition is provided wherein the ratios of a multi active agent, an unctuous emollient and an added polar solvent as penetration enhancer are selected or adapted to provide a selected pharmacological or safety property;

In one or more embodiments a foamable pharmaceutical composition is provided also incorporating a polymeric agent.

In one or more embodiments the polymeric agent is selected from a bioadhesive agent, a gelling agent, a film forming agent and a phase change agent and can be from about 0.01% to about 5% by weight.

In one or more embodiments of the pharmaceutical or cosmetic foamable product is non-flammable.

Water and optional ingredients are added to complete the total mass to 100%.

All % values are provided on a weight (w/w) basis.

### Multi-Active Agent or Component

A Multi-Active Agent or Component is an agent that whilst having an emulsifying like effect with an unctuous emollient may also have in addition to some extent one or more of the properties of foam adjuvant, friction ameliorator, gelling agent, look and feel ameliorator, lubricant, stabilizer, anti-destabilizer, surfactant, thickener and viscosity modifier or enhancer.

In one embodiment the multi active agent may help to ameliorate, counteract, or overcome undesirable effects and drawbacks of using an unctuous emollient.

In one or more embodiments the multi-active agent can be, a surfactant system comprising of a surfactant and a co surfactant, a waxy emulsifier, a liquid wax, a liquid crystal emulsifier, an emulsifier which is solid or semi solid at room temperature and pressure, an emulsifier which is a combination of a solid/semi solid agent and a liquid agent, an emulsifier which is a combination of two or more liquid agents or combinations of two or more agents in an appropriate proportion as will be appreciated a person skilled in the art.

In one or more embodiments the multi-active agent is a semi solid or solid at RTP, for example TPGS (alpha-tocopheryl polyethylene glycol succinate) or polyoxyethylene alkyl ethers.

In one or more embodiments the multi-active agent is a complex emulgator in which the combination of two or more emulgators provides a more stable emulsion or improved foam quality than a single emulgator. For example and by way of non-limiting explanation alone it has been found that by choosing say two emulgators one hydrophobic and the other hydrophilic the combination can produce a more stable emulsion than a single emulgator. Preferably, the complex emulgator has a combination of emulgators wherein there is a difference of about 4 or more units between the HLB values of two of the emulgators or there is a significant difference in the chemical nature or structure of two of the emulgators. In some cases the difference is about at least 8; and in other embodiments is about at least 10, for example steareth 2 and steareth 21; or more. In certain circumstances the complex emulgator can be a combination of a surfactant which can by itself be capable of producing an emulsion and a foam adjuvant, which can stabilize the emulsion and boost the production of foam, for example where one of the surfactants is ceteth 10 and the foam adjuvant is for example, behenyl alcohol, which has a low HLB; or the surfactant is say span 80 or steareth 2, which have low HLB's and the foam adjuvant is cetearyl alcohol which has a high required HLB.

In one or more embodiments the multi-active agent comprises a two or more surfactants with a HLB below about 13. In one or more other embodiments, the multi-active agent comprises a surfactant with a HLB below about 9. In one or more further embodiments the multi-active agent comprises a two or more surfactants with a mean HLB below about 9. In one or more other embodiments the multi-active agent comprises at least one surfactant and at least one foam adjuvant or cosurfactant, wherein the surfactant has a HLB below about 9. In one or more further embodiments the surfactant of the multi active agent has a HLB within 2 units of the required HLB of the oil phase. In one or more further embodiments the surfactant of the multi active agent has a HLB within 1 unit of the required HLB of the oil phase.

In one or more embodiments, the multi-active agent can be, a cocktail of a surfactant system and a polymer or a polymeric agent; more specifically it can be a cocktail of a surfactant system and a metal starch; of a surfactant system and a hydrophobic starch; a cocktail of a surfactant system and a microcrystalline cellulose; a cocktail of a surfactant system and a cellulose ether and or long chain polysaccharide; a cocktail of a surfactant system and TPGS (alpha-tocopheryl polyethylene glycol succinate); and a cocktail of a surfactant system and crosslinked polyacrylic acid polymers and the like. Specific examples of multi active agent cocktail systems are exemplified in the Examples. Complex emulgators include sucrose stearate and arlacel; glyceryl monostearate and ceteth 10; cetearyl glucoside and sorbitan stearate.

In one or more embodiments, the multi active agent is selected from the group consisting of
(a) two or more complex emulgators wherein there is a difference of about 4 or more units between the HLB values of two of the emulgators or there is a significant difference in the chemical nature or structure of two of the emulgators;
(b) a surfactant and a foam adjuvant or co surfactant, wherein the surfactant has a HLB close to the required HLB of the oil phase;
(c) a surfactant and a liquid wax, wherein the surfactant has a HLB close to the required HLB of the oil phase;
(d) a surfactant and a polymeric agent other than starch or a modified starch ester, wherein the surfactant has a HLB close to the required HLB of the oil phase;
(e) a polymeric agent and a foam adjuvant or co surfactant, which can cooperate to stabilize the emulsion;
(f) a single surfactant without a long polymeric side chain that is composed of a mixture of esters having a HLB close to the required HLB of the oil phase;
(g) combinations of any of the above;

Specific non limiting examples of a multi active agent surfactant systems are, combinations of polyoxyethylene alkyl ethers, such as Brij 59/Brij10; Brij 52/Brij 10; Stearath 2/Stearath 20; Stearath 2/Stearath 21 (Brij 72/BRIJ 721); Myrj 52/Myrj 59; combinations of sucrose esters, such as Surphope 1816/Surphope 1807; combinations of sorbitan esters, such as Span 20/Span 80; Span 20/Span 60; combinations of sucrose esters and sorbitan esters, such as Surphope 1811 and Span 60; combinations of liquid polysorbate detergents and PEG compounds, such as Twin 80/PEG-40 stearate/; methyl glucasol sequistearate; polymeric emulsifiers, such as Permulen (TR1 or TR2); liquid crystal systems, such as Arlatone (2121), Stepan (Mild RM1), Nikomulese (41) and Montanov (68); ceteth-20, span 80 and a foam adjuvant/cosurfactant like behenyl alcohol; ceteth-20 and span 80; polysorbate 80 and span 80; sucrose stearic acid esters; polysorbate 80 and steareth 2; span 20 and a liquid wax like isostearic acid or oleyl alcohol; ceteth 20 and a foam adjuvant/cosurfactant like behenyl alcohol; ceteth 20, a foam adjuvant/cosurfactant like behenyl alcohol and polysorbate 60; ceteth-10, span 80 and a foam adjuvant/cosurfactant like behenyl alcohol; ceteth-10, span 20 and a foam adjuvant/cosurfactant like behenyl alcohol; span 80, span 20 and laureth-4; sorbitan oleate and polysorbate 60; methocel and xanthan gum; sorbitan monopalmitate (which is a mixture of esters ideally suitable for petrolatum emulsions); a surfactant such as span20 or span 40 and an emollient foam adjuvant such as cetearyl alcohol; an and the like. Indeed sucrose stearic acid esters (mono, di and tri) Surfhope D-1807 any of these combinations may benefit at least to some extent from the addition and incorporation of a an emollient foam adjuvant. Uniquely, it has been found that sorbitan fatty acid esters (span 20, 40; 60; and 80) are surprisingly suitable as sole agent or in combination with an emollient foam adjuvant for working with and miscible in petroleum foam formulations—and without being bound by any particular theory or suggestion—this advantage apart from having a generally suitable HLB (all being below 9) and the selection of one which provides a HLB close to the required HLB of the oil phase it may possibly be without being bound by any particular theory be due to one or more of the sugar moiety coupled to a relatively medium short fatty acid chain; the absence of a long polymeric side chain found in various other surfactants; the compact size of the surfactant, which may facilitate dissolution and a stabilizing interaction with one or more other substances in the composition; and that it comprises a mixture if esters. Similar reasoning may apply to the use of Surfhope, which is a mixture of mono, di and tri sucrose stearic acid esters.

In one or more embodiments the multi-active agent is selected from the group consisting of combinations of polyoxyethylene alkyl ethers, Brij 59/Brij10; Brij 52/Brij 10; Stearath 2/Stearath 20; Stearath 2/Stearath 21 (Brij 72/BRIJ 721); Myrj 52/Myrj 59; combinations of sucrose esters, such as Surphope 1816/Surphope 1807; combinations of sorbitan esters; Span 20/Span 80; Span 20/Span 60; combinations of sucrose esters and sorbitan esters, Surphope 1811 and Span 60; combinations of liquid polysorbate detergents and PEG compounds, Twin 80/PEG-40 stearate/methyl glucose sequistearate; ceteth-20 and span 80; polysorbate 80 and span 80; polysorbate 80 and steareth 2; span 80, span 20 and laureth-4; ceteth 20 and polysorbate 60; sorbitan oleate and polysorbate 60; a foam adjuvant or cosurfactant and any of the following: span 20; span 40; span 60; span 80; ceteth-20; Permulen (TR1 or TR2) a polymeric emulsifier; Arlatone (2121), Stepan (Mild RM1), Nikomulese (41) and Montanov (68); ceteth-20, span 80 and a foam adjuvant/cosurfactant; ceteth 20 and behenyl alcohol; a foam adjuvant and emulgators, behenyl alcohol, ceteth 20 and polysorbate 60; ceteth-10, span 80 and a foam behenyl alcohol; ceteth-10, span 20 and behenyl alcohol; a foam adjuvant and a polymeric agent methocel and xanthan gum or carboxymethylcellulose sodium; sucrose stearic acid esters; sorbitan fatty acid esters with or without cetearyl alcohol; span 20; or span 40 and a liquid wax; span 20; or span 40 and isostearic acid or oleyl alcohol;

### Unctuous Emollient

A “unctuous emollient” as used herein refers to a greasy, fatty, waxy or oily material, including liquids, semi solids and solids

Non limiting examples of unctuous emollients that may be used in the pharmaceutical composition of the present invention may be natural or synthetic or a synthetic derivative and, include higher aliphatic hydrocarbons, animal or vegetable fats, greases and oils, waxes, and combinations thereof.

In one or more embodiments, specific non limiting examples of the higher aliphatic hydrocarbons include petrolatum including white petrolatum, yellow petrolatum, soft petrolatum, vaseline, vaseline jelly, mineral jelly and fractions thereof, paraffin, squalane, ceresin, mineral oil and the like.

In one or more embodiments, specific non limiting examples of the waxes include beeswax, carnauba wax, microcrystalline wax, candililla wax, berry wax, montan wax, polyethylene wax and ethylene vinyl acetate (EVA) copolymers spermaceti, lanolin, wool wax, wool fat, wax blend, solid paraffin, oxidized wax, waxy solids or waxy semi-solids, synthetic wax's and the like.

In one or more embodiments, non limiting specific examples of the animal or vegetable fats and oils include, triglycerides, olive oil, almond oil, avocado oil, borage oil, castor oil, cocoa butter, palm oil, turtle oil, cod-liver oil, whale oil, beef tallow, butter fat, shea butter, shorea butter, and the like.

In one or more embodiments, the above-described unctuous emollient substances may be used alone or in combination.

In one or more embodiments, the unctuous emollient is a high-melting point hydrocarbon, such as, petrolatum.

The use of high melting point hydrocarbons, such as petrolatum in high concentrations of preferably more than 20% are not always desirable since they can be occlusive when applied to the skin. On the other hand, for example embodiments, between about 1% to about 10% or to about 20% or sometimes more depending on the composition may not be occlusive or merely partially or temporarily occlusive and are included in the composition of the present invention; yet, in certain additional embodiments, when an extensive refatting or moisturizing effect is required, then petrolatum in concentrations of more than 10%, for example between about 10% and about 49% is included in the composition of the present invention.

Surprisingly, the basic occlusive nature of high levels of petrolatum can be ameliorated or retarded by using certain levels of oil combined with petrolatum; or by using certain levels of liquid wax combined with petrolatum or certain levels of foam adjuvant/co-surfactant combined with petrolatum; or certain levels of oil combined with foam adjuvant and petrolatum; or combinations with petrolatum including a thinning agent being an agent that is compatible and miscible with petrolatum and which can substantially reduce the viscosity of the pre foam formulation. Low viscous liquid aliphatic hydrocarbons may be suitable.

In order to derive, develop or optimize a composition, which is readily foamable upon release from a pressurized container, additional components may also be introduced, as provided herein below.

### Liquid Wax

A wax can be a solid wax or a liquid wax. For the purposes herein wax includes waxy substances, like fatty acids and their fatty alcohol counterparts, which can be short, medium and long chain. The fatty acid or alcohol backbone may be straight, branched, saturated, unsaturated, or hydrogenated, unhydrogenated, natural, or synthetic. Where one type of backbone produces a waxy substance then molecules with the substantially the same backbone are also deemed as being part of the wax family or being a waxy substance counterpart or derivative thereof. For example, stearic acid, which is a waxy solid, has a C18 backbone. In other cases where the carbon backbone chain is 18 C, such as stearyl alcohol a solid and isostearic acid, oleic acid and oleyl alcohol, which are liquids, are all considered to be waxy substances, having a commonality with regards to the number of carbon atoms in the formula. Also within the scope is where hydrogenation would form a wax or waxy substance. In a preferred embodiment the wax is a liquid wax. Liquid waxes can in an embodiment help to thin or reduce the viscosity of the pre-foam formulations. They can also improve the sensory qualities and look and feel of the resultant foam. Non limiting examples of liquid waxes are oleyl alcohol, isostearyl alcohol, capric alcohol, capryl alcohol, isostearic acid, caprylic acid, caproic acid, and butyric acid, and also jojoba oil.

Jojoba oil (pronounced “ho-HO-bah”) is the liquid wax produced in the seed of the Jojoba (Simmondsia chinensis) plant. Jojoba oil is a straight chain wax ester, 36 to 46 carbon atoms in length. Each molecule consists of a fatty acid and a fatty alcohol joined by an ester bond. Each molecule has two points of cis-unsaturation, both located at the 9th carbon atom from either end of the molecule. Jojoba oil comprises approximately 66-71% eicosenoic acid, 14-20% docosenoic acid and 10-13% oleic acid. Refined jojoba oil is colorless and odorless. The melting point of jojoba oil is approximately 10° C. Jojoba oil is relatively shelf-stable when compared with other vegetable oils. Unlike common vegetable oils, jojoba oil is chemically very similar to human sebum. Therapeutically it can aid in the healing process.

Sebum acts to protect and waterproof hair and skin, and keep them from becoming dry, brittle and cracked. It can also inhibit the growth of microorganisms on skin. It is thought that likewise, formulations with substances such as waxes that can mimic sebum for example like jojoba oil, stearic acid, isostearic acid, oleyl alcohol and the like including combinations thereof and especially in higher concentrations can provide protection to the hair and skin. Moreover without being bound by any theory, the application of formulations containing jojoba might create a feed back control cycle and result in the amelioration any over or under production of sebum or assist in the cleaning of blocked pores. Thus, it may be useful in combination with an anti acne preparation. In one or more embodiments it is used with a coal tar extract. In other embodiments it may be used with benzyl peroxide (BPO).

In one or more embodiments, the fatty acid or alcohol is a biologically active. For example, benhenyl alcohol has some antiviral properties apart from being a foam adjuvant or co-surfactant. In an embodiment, biologically active fatty acid or alcohol possesses keratolytic activities.

In an embodiment of the present invention, the waxy substance is incorporated in the foamable composition in a safe and effective amount. The term “safe and effective” means an amount of an active agent that exerts a therapeutic effect on a specific disorder, without causing adverse effects that may prohibit the use of said active agent in the treatment of said disorder.

In one or more embodiments, the wax, waxy substance, counterpart or derivative thereof contributes to the foam structure.

### Hydrophobic Solvents

Further in one or more embodiments the unctuous emollients of the present invention may also be combined with one or more hydrophobic solvents or carriers, which are materials suitable for use to blend with or act as a carrier for the unctuous emollients. They may also have a further role in effecting the solubility of an API.

In one or more other embodiments the hydrophobic solvents or carriers are ester oils. Specific non limiting examples of the ester oils include isopropyl myristate, isopropyl palmitate, butyl stearate, hexyl laurate, octyldodecyl myristate, di-isopropyl adipate, isocetyl myristate, di-isopropyl sebacate, and the like.

In one or more other embodiments the hydrophobic solvents or carriers are higher alcohols. Specific non limiting examples of the higher alcohols include cetyl alcohol, oleyl alcohol, isostearyl alcohol, octyldodecanol and the like.

According to one or more embodiments, hydrophobic solvents or carriers are liquid oils originating from vegetable, marine or animal sources. Suitable liquid oil includes saturated, unsaturated or polyunsaturated oils. By way of example, the unsaturated oil may be olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion. Other non limiting oil examples are palm oil, coconut oil and tallow.

Suitable hydrophobic solvents or carriers also include polyunsaturated oils containing poly-unsaturated fatty acids. In one or more embodiments, the unsaturated fatty acids are selected from the group of omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Such unsaturated fatty acids are known for their skin-conditioning effect, which can contribute to the therapeutic benefit of the present foamable composition. Thus, the hydrophobic solvent can include at least 3% preferably at least 6% of an oil selected from omega-3 oil, omega-6 oil, and mixtures thereof.

In the context of the present invention, oils that possess therapeutically beneficial properties are termed as “therapeutically active oil.”

Another class of hydrophobic solvents or carriers is the essential oils, which are also considered therapeutically active oils, and which contain active biologically occurring molecules and, upon topical application, exert a therapeutic effect. Non-limiting examples of essential oils include rosehip oil, which contain retinoids and is known to reduce acne and post-acne scars, and tea tree oil, which possess antibacterial, antifungal and antiviral properties. Other examples of essential oils are oils of anise, basil, bergemont, camphor, cardamom, carrot, canola, cassia, catnip, cedarwood, citronella, clove, cypress, eucalyptus, frankincense, garlic, ginger, grapefruit, hyssop, jasmine, jojova, lavender, lavandin, lemon, lime, mandarin, marjoram, myrrh, neroli, nutmeg, orange, peppermint, petitgrain, rosemary, sage, spearmint, star anise, tangerine, thyme vanilla, verbena and white clover.

Another class of therapeutically active oils includes liquid hydrophobic plant-derived oils, which are known to possess therapeutic benefits when applied topically.

Silicone oils also may be used and are desirable due to their known skin protective and occlusive properties. Suitable silicone oils include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. Silicone oils are also considered therapeutically active oil, due to their barrier retaining and protective properties.

A further class of hydrophobic solvents or carriers includes hydrophobic liquids, selected from the family of organic liquids described as “emollients.” Emollients possess a softening or soothing effect, especially when applied to body areas, such as the skin and mucosal surfaces. Examples of suitable emollients include isopropyl myristate, isopropyl palmitate, isopropyl isostearate, diisopropyl adipate, diisopropyl dimerate, maleated soybean oil, octyl palmitate, cetyl lactate, cetyl ricinoleate, tocopheryl acetate, cetyl acetate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, octyl dodecanol, sucrose esters of fatty acids and octyl hydroxystearate.

The foamable composition of the present invention can be an emulsion, or microemulsion, or nanoemulsion including an aqueous phase and an organic carrier phase.

One non-limiting benefit of combining a multi active agent and a unctuous emollient is apparent in the resulting conservation of skin barrier properties.

Another non-limiting benefit of combining a multi active agent and a unctuous emollient is further apparent in the reduction of skin irritation.

Another non-limiting benefit of the vehicle or composition of the present invention is to provide satisfactory or increased penetration of the active or beneficial agent whilst replenishing the skin for example by moisturizing or adding fats or oils.

The ratio between the multi-active agent and the unctuous emollient is determined according to the desirable level of unctuous emollient and taking into account appropriate and desirable pharmacologic and safety properties of the product. Typically, the multi-active agent to unctuous emollient ranges between about 1:1 and about 1:20, for example, about 1:1, about 2:5, about 1:5, about 2:15, about 1:10, about 2:25, about 1:15, about 2:35, about 1:20, about 2:45 and about 1:25, preferably between about 2:5 to 2:35.

Where the unctuous emollient is a combination of petrolatum and an oil the ratio between petrolatum and the oil is determined according to the desirable level of unctuous emollient and taking into account appropriate and desirable pharmacologic and safety properties of the product. Typically, the ratio of oil to petrolatum ranges between about 1:6 and about 6:1, for example, about 1:6, about 5:27, about 1:5, about 1:4, about 1:3, about 3:7, about 1:2, about 1:1, about 2:1, about 7:3, about 3:1, about 4:1 and about 5:1, about 27:5, and about 1:6 preferably between about 1:4 to about 2:1 and more preferably between about 1:3 and about 1:2.

Where the unctuous emollient is a combination of petrolatum, an oil and an emollient foam adjuvant the ratio between the unctuous emollient and the emulsifiying agent (excluding foam adjuvants/co-surfactants) is typically, in excess of 1:8; for example being about or in excess of any of the following about 1:9; or about 1:10; or about 1:11; or about 1:12; or about 1:13; or about 1:14; or about 1:15;; or about 1:16; or about 1:17; or about 1:18; or about 1:19; or about 1:20; or about 1:21; or about 1:22; or about 1:23; or about 1:24; or about 1:25; or about 1:30; or about 1:35; or about 1:40.

Where the unctuous emollient is a combination of petrolatum and a liquid wax the ratio between petrolatum and the liquid wax typically, ranges between about 1:4 to about 10:1, for example, about 1:3; or about 1:2; or about 1:1; or about 2:1; or about 3:1; or about 4:1; or about 5:1; or about 6:1; or about 7:1; or about 8:1; or about 9:1; or about 10:1 and preferably between about 1:1 and about 4:1.

Where the unctuous emollient is a combination of petrolatum and an emollient foam adjuvant the ratio between petrolatum and the foam adjuvant typically, ranges between about 70:1 to about 2:1, for example, about 60:1; or about 30:1; or about 15:1; or about 10:1; or about 8:1 or about 5:1; or about 3:1; or about 5:2; and preferably between about 30:1 and about 8:1.

### Surface Active Agent

Surface-active agents (also termed “surfactants”) include any agent linking oil and water in the composition, in the form of emulsion. A surfactant's hydrophilic/lipophilic balance (HLB) describes the emulsifier's affinity toward water or oil. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). The surface-active agent according to the present invention has an HLB value, suitable for stabilizing an emulsion comprising the aqueous phase and the unctuous emollient of the composition. HLB is of more significance with non-ionic surfactants.

According to one or more embodiments of the present invention, the surface-active agent has a hydrophilic lipophilic balance (HLB) between about 9 and about 14, which is the required HLB (the HLB required to stabilize an O/W emulsion of a given oil) of most oils and hydrophobic solvents. Thus, in one or more embodiments, the composition contains a single surface active agent having an HLB value between about 9 and about 14 (e.g., about 9, about 10, about 11, about 12, about 13 and about 14), and in one or more embodiments, the composition contains more than one surface active agent and the weighted average of their HLB values is between about 9 and about 14 (e.g. about 9, about 10, about 11, about 12, about 13 and about 14). Yet, in other embodiments, when a water-in-oil emulsion is desirable, the composition contains one or more surface-active agents, having an HLB value between about 2 and about 9 (e.g., about 2, about 3, about 4, about 5, about 6, about 7, about 8 and about 9).

It should be noted that HLB values may not be so applicable to non ionic surfactants, for example, with liquid crystals or with silicones.

The surface-active agent is selected from anionic, cationic, nonionic, zwitterionic, amphoteric and ampholytic surfactants, as well as mixtures of these surfactants. Such surfactants are well known to those skilled in the therapeutic and cosmetic formulation art. Non-limiting examples of possible surfactants include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49, Myrj 52 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 21, brij 721, brij 38, brij 52, brij 56 and brij W1; sucrose esters, partial esters of sorbitol and its anhydrides, such as sorbitan monolaurate and sorbitan monolaurate; mono or diglycerides, isoceteth-20, sodium methyl cocoyl taurate, sodium methyl oleoyl taurate, sodium lauryl sulfate, triethanolamine lauryl sulfate and betaines.

In one or more embodiments of the present invention, the surface-active agent comprises a non-ionic surfactant since ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone provide foams of excellent quality, i.e. a score of “E” according to the grading scale discussed herein below.

In one or more embodiments, the surface-active agent includes a mixture of a non-ionic surfactant and an ionic surfactant in a ratio in the range of about 100:1 to about 6:1. In one or more embodiments, the non-ionic to ionic surfactant ratio is greater than about 6:1, or greater than about 8:1; or greater than about 14:1, or greater than about 16:1, or greater than about 20:1.

In one or more embodiments of the present invention, a combination of a non-ionic surfactant and an ionic surfactant (such as sodium lauryl sulphate and cocamidopropylbetaine) is employed, at a ratio of between 1:1 and 20:1, for example, about 1:1, about 4:1, about 8:1, about 12:1, about 16:1 and about 20:1 or at a ratio of 4:1 to 10:1, for example, about 4:1, about 6:1, about 8:1 and about 10:1.

Thus, in an exemplary embodiment, a combination of an non-ionic surfactant having HLB of less than about 9 and an non-ionic surfactant having HLB of equal or more than about 9 is employed, at a ratio of between about 1:8 and about 8:1, or at a ratio of about 4:1 to about 1:4, wherein the HLB of the combination of emulsifiers is between about 5 and about 18.

In one or more embodiments of the present invention, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

The total surface-active agent is in the range of about 0.1% to about 15% of the composition, preferably is about 0.5% to about 10% and is occasionally less than about 2% or less than about 1%.

### Heumectant

A heumectant, is a substance that helps retain moisture and also prevents rapid evaporation. Non limiting examples are propylene glycol, propylene glycol derivatives, glycerin, hydrogenated starch hydrosylate, hydrogenated lanolin, lanolin wax, D manitol, sorbitol, sodium 2-pyrrolidone-5-carboxylate, sodium lactate, sodium PCA, soluble collagen, dibutyl phthalate, and gelatin. Other examples may be found in the Handbook of Pharmaceutical Additives published by Gower.

### Moisturizers

A moisturizer, is a substance that helps retain moisture or add back moisture to the skin. Examples are allantoin, petrolatum, urea, lactic acid, sodium PCV, glycerin, shea butter, caprylic/capric/stearic triglyceride, candelilla wax, propylene glycol, lanolin, hydrogenated oils, squalene, sodium hyaluronate and lysine PCA. Other examples may be found in the Handbook of Pharmaceutical Additives published by Gower.

Pharmaceutical compositions of the present invention may in one or more embodiments usefully comprise in addition a heumectant or a moisturizer or combinations thereof.

### Polar Solvent

A “polar solvent” is an organic solvent, typically soluble in both water and oil. Certain polar solvents, for example propylene glycol and glycerin, possess the beneficial property of a heumectant.

In one or more embodiments, the polar solvent is a heumectant.

In one or more embodiments, the polar solvent is a polyol. Polyols are organic substances that contain at least two hydroxy groups in their molecular structure.

In one or more embodiments, the polar solvent contains an diol (a compound that contains two hydroxy groups in its molecular structure), such as propylene glycol (e.g., 1,2-propylene glycol and 1,3-propylene glycol), butanediol (e.g., 1,4-butanediol), butanediol (e.g., 1,3-butanediol and 1,4-butenediol), butynediol, pentanediol (e.g., 1,5-pentanediol), hexanediol (e.g., 1,6-hexanediol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the polar solvent contains a triol (a compound that contains three hydroxy groups in its molecular structure), such as glycerin and 1,2,6-Hexanetriol.

Other non-limiting examples of polar solvents include pyrrolidones, (such as N-methyl-2-pyrrolidone and 1-methyl-2-pyrrolidinone), dimethyl isosorbide, 1,2,6-hexapetriol, dimethyl sulfoxide (DMSO), ethyl proxitol, dimethylacetamide (DMAc) and alpha hydroxy acids, such as lactic acid and glycolic acid.

According to still other embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

Polar solvents are known to enhance the penetration of active agent into the skin and through the skin, and therefore, their inclusion in the composition of the present invention can be desirable, despite their undesirable skin drying and irritation potential. There is at one level a commonality between the different polar solvents and their penetration enhancement properties. Lower molecular weight alcohols can sometimes be more potent as a solvent, for example by extracting lipids from the skin layers more effectively, which characteristic can adversely affect the skin structure and cause dryness and irritation. Therefore the selection of lower molecular weight alcohols is ideally avoided.

### Potent Solvent

In one or more embodiments of the present invention, the foamable composition includes a potent solvent, in addition to or in place of one of the hydrophobic solvents, polar solvents or emollients of the composition. A potent solvent is a solvent other than mineral oil that solubilizes a specific active agent substantially better than a hydrocarbon solvent such as mineral oil or petrolatum. For example, a potent solvent solubilizes the active agent 5 fold better than a hydrocarbon solvent; or even solubilizes the active agent 10-fold better than a hydrocarbon solvent.

In one or more embodiments of the present invention, the composition includes at least one active agent in a therapeutically effective concentration; and at least one potent solvent in a sufficient amount to substantially solubilize the at least one active agent in the composition. The term “substantially soluble” means that at least 95% of the active agent has been solubilized, i.e., 5% or less of the active agent is present in a solid state. In one or more embodiments, the concentration of the at least one potent solvent is more than about 40% of the at least one solvent of the composition of the present invention; or even more than about 60%.

Non-limiting examples of pairs of active agent and potent solvent include:

Betamethasone valerate: Practically insoluble in mineral oil (<0.01%); soluble more than 1% in glycofurol;

Hydrocortisone butyrate: Practically insoluble in mineral oil (<0.01%); soluble more than 1% in glycofurol;

Metronidazole: Practically insoluble in mineral oil (<0.01%); soluble more than 1% in dimethyl isosrbide;

Ketoconazole: Practically insoluble in mineral oil (<0.01%); soluble more than 1% in glycofurol, propylene glycol and dimethyl isosrbide;

Mupirocin: Practically insoluble in mineral oil (<0.01%); soluble more than 1% in glycofurol, hexylene glycol, dimethyl isosorbide, propylene glycol and polyethylene glycol 400 (PEG 400);

Meloxicam, a nonsteroidal anti-inflammatory agent: Practically insoluble in mineral oil (<0.001%); soluble in propylene glycol: 0.3 mg/mL; and in PEG 400: 3.7 mg/mL; and

Progesterone: Practically insoluble in mineral oil (<0.001%); soluble in PEG 400: 15.3 mg/mL.

A non-limiting exemplary list of solvents that can be considered as potent solvents includes polyethylene glycol, propylene glycol, hexylene glycol, butanediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, such as dimethyl isosorbide, glycofurol and ethoxydiglycol (transcutol).

In another aspect, the present invention provides a method of designing a stable unctuous foamable composition by selecting at least one active agent; and identifying a solvent that solubilizes the active agent substantially better than mineral oil or petrolatum, for example, solubilizes the active agent 5-fold better or even 10-fold better than a hydrocarbon solvent such as mineral oil or petrolatum. The method may further include adjusting the type and concentration of surfactant and gelling agent to provide a foamable composition.

In another aspect of the present invention the active agent has a degree of solubility in water, in petrolatum, in the emulsion or a phase thereof and a potent solvent is used to increase the solubility, in one or both phases, in the interphase or in the foam.

In another aspect of the present invention the active agent has a limited degree of solubility in water, in petrolatum, in the emulsion or a phase thereof and a potent solvent is used to increase the solubility, in one or both phases, in the interphase or in the foam.

The use of a potent solvent in a foam composition provides an improved method of delivering poorly soluble therapeutic agents to a target area. It is known that low drug solubility results in poor bioavailability, leading to decreased effectiveness of treatment. Foam compositions of the present invention, for which the solvent includes a potent solvent, increase the levels of the active agent in solution and thus, provide high delivery and improved therapy.

Potent solvents, as defined herein, are usually liquid. Formulations comprising potent solvents and active agents are generally disadvantageous as therapeutics, since their usage involves unwanted dripping and inconvenient method of application; resulting in inadequate dosing. Surprisingly, the foams of the present invention, which are drip-free, provide a superior vehicle for such active agents, enabling convenient usage and accurate effective dosing.

In one or more embodiments of the present invention the present invention the foamable pharmaceutical composition may additionally include a potent solvent or a mixture of two or more of the above solvents selected from the group of hydrophobic solvents, silicone oils, emollients, polar solvents and potent solvents in an appropriate proportion as would be appreciated to a person skilled in the art.

### Polymeric Agent

In one or more embodiments, the foamable composition contains a polymeric agent. The polymeric agent serves to stabilize the foam composition and to control drug residence in the target organ.

In one or more specific non limiting embodiments, the polymeric agent is ASOS, carboxymethyl cellulose/microcrystalline cellulose, Arlacel 2121, or methocel and xantham gum.

More exemplary polymeric agents are classified below in a non-limiting manner. In certain cases, a given polymer can belong to more than one of the classes provided below.

In one or more embodiments, the composition of the present invention includes a gelling agent. A gelling agent controls the residence of a therapeutic composition in the target site of treatment by increasing the viscosity of the composition, thereby limiting the rate of its clearance from the site. Many gelling agents are known in the art to possess mucoadhesive properties.

The gelling agent can be a natural gelling agent, a synthetic gelling agent and an inorganic gelling agent. Exemplary gelling agents that can be used in accordance with one or more embodiments of the present invention include, for example, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, starch, chemically modified starches and the like, semi-synthetic polymeric materials such as cellulose ethers (e.g. hydroxyethyl cellulose, hydroxypropyl cellulose, methyl cellulose, carboxymethyl cellulose (CMC), methylhydroxyethylcellulose, methylhydroxypropylcellulose, microcrystalline cellulose with CMC, hydroxypropylmethyl cellulose, hydroxyethylcarboxymethylcellulose, carboxymethylcellulose and carboxymethylhydroxyethylcellulose), guar gum, hydroxypropyl guar gum, soluble starch, cationic celluloses, cationic guars, and the like, and synthetic polymeric materials, such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like. Mixtures of the above compounds are also contemplated.

Further exemplary gelling agents include the acrylic acid/ethyl acrylate copolymers and the carboxyvinyl polymers. Non-limiting examples include Carbopol® 934, Carbopol® 940, Carbopol® 950, Carbopol® 980, Carbopol® 951 and Carbopol® 981. Such agents can function as stabalisers in one or more embodiments of the present invention and as delivery enhancers in one or more other embodiments of the present invention.

Yet, in other embodiments, the gelling agent includes inorganic gelling agents, such as silicone dioxide (fumed silica).

Mucoadhesive/bioadhesion has been defined as the attachment of synthetic or biological macromolecules to a biological tissue. Mucoadhesive agents are a class of polymeric biomaterials that exhibit the basic characteristic of a hydrogel, i.e. swell by absorbing water and interacting by means of adhesion with the mucous that covers epithelia. Compositions of the present invention may contain a mucoadhesive macromolecule or polymer in an amount sufficient to confer or partially to confer bioadhesive properties, although these substances may by their nature, increase the tackiness of a composition so this will be taken into account in preparing compositions of the present invention. The bioadhesive macromolecule can enhance delivery of biologically active agents on or through the target surface. The mucoadhesive macromolecule may be selected from acidic synthetic polymers, preferably having an acidic group per four repeating or monomeric subunit moieties, such as poly(acrylic)-and/or poly(methacrylic) acid (e.g., Carbopol®, Carbomer®), poly(methylvinyl ether/maleic anhydride) copolymer, and their mixtures and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts, usually in a final concentration of about 0.01% to about 0.5% by weight. Many mucoadhesive agents are known in the art to also possess gelling properties.

In one or more embodiments, the polymeric agent contains a film-forming component, although these substances may also by their nature, increase the tackiness of a composition so this will be taken into account in preparing compositions of the present invention. The film-forming component may include a water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross-linking agent may be used to modify the polymer's characteristics. For example, esters such as dibutyl or diethyl phthalate, amides such as diethyldiphenyl urea, vegetable oils, fatty acids and alcohols such as oleic and myristyl acid may be used in combination with the cellulose derivative.

In one or more embodiments, the polymeric agent includes a phase change polymer, which alters the composition behavior from fluid-like prior to administration to solid-like upon contact with the target mucosal surface. Such phase change results from external stimuli, such as changes in temperature or pH and exposure to specific ions (e.g., Ca²⁺). Non-limiting examples of phase change polymers include poly(N-isopropylamide) and Poloxamer 407®.

It has been discovered also that by using a derivatized hydrophilic polymer with hydrophobic alkyl moieties as a polymeric emulsifier such as pemulen it is possible to stabilize the emulsion better particularly about or at the region of phase reversal tension. It can be used with a true surfactant or with a foamadjuvant or cosurfactant to good effect. Although a polymer it also has emulsifying qualities. Other types of derivatized polymers like silicone copolymers, derivatized starch [Aluminum Starch Octenylsuccinate (ASOS)]/[DRY-FLO AF Starch], and derivatized dexrin may also a similar stabilizing effect.

A series of dextrin derivative surfactants prepared by the reaction of the propylene glycol polyglucosides with a hydrophobic oxirane-containing material of the glycidyl ether are highly biodegradable. [Hong-Rong Wang and Keng-Ming Chen, Colloids and Surfaces A: Physicochemical and Engineering Aspects Volume 281, Issues 1-3, 15 Jun. 2006, Pages 190-193]. It is thought such dextrin derivatives may also be useful.

The polymeric agent is present in an amount in the range of about 0.01% to about 5.0% by weight of the foam composition. In one or more embodiments, it is typically less than about 1 wt % of the foamable composition.

### Foam Adjuvant

Preferably, a therapeutically effective foam adjuvant is included in the foamable compositions of the present invention to increase the foaming capacity of surfactants and/or to stabilize the foam. In one or more embodiments of the present invention, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains. Foam adjuvants, as defined herein are also useful in facilitating improved spreadability and absorption of the composition.

In one or more embodiments of the present invention, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

In one or more embodiments, a combination of a fatty acid and a fatty ester is employed.

Optionally, the carbon atom chain of the fatty alcohol or the fatty acid may have a double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

A property of the fatty alcohols and fatty acids used in context of the composition of the present invention is related to their therapeutic properties per se. Long chain saturated and mono unsaturated fatty alcohols, e.g., stearyl alcohol, erucyl alcohol, arachidyl alcohol and behenyl alcohol (docosanol) have been reported to possess antiviral, antiinfective, antiproliferative and anti-inflammatory properties. Longer chain fatty alcohols, e.g., tetracosanol, hexacosanol, heptacosanol, octacosanol, triacontanol, etc., are also known for their metabolism modifying properties and tissue energizing properties. Long chain fatty acids have also been reported to possess anti-infective characteristics.

Behenyl alcohol is saturated C22 fatty alcohol, which apart from having antiviral activity and acting as a co-surfactant or foam adjuvant is said to usable as a thickening agent and can, help make skin smoother and prevent moisture loss. Cetearyl Alcohol is a waxy mixture of fatty alcohols, being primarily cetyl and stearyl alcohols. It is used as an emulsion stabilizer, foam booster, and viscosity-increasing agent and it imparts an emollient feel to the skin.

In one or more embodiments, the active agent is encapsulated in particles, microparticles, nanoparticles, microcapsules, spheres, microspheres, nanocapsules, nanospheres, liposomes, niosomes, polymer matrix, nanocrystals or microsponges.

The composition of the present invention may further optionally include a variety of formulation excipients, which are added in order to fine-tune the consistency of the formulation, protect the formulation components from degradation and oxidation and modify their consistency. Such excipients may be selected, for example, from stabilizing agents, antioxidants, humectants, moisturizers, preservatives, colorant and odorant agents and other formulation components, used in the art of formulation.

### Propellant

Aerosol propellants are used to generate and administer the foamable composition as a foam. The total composition including propellant, foamable compositions and optional ingredients is referred to as the foamable carrier. The propellant makes up about 3% to about 25% (w/w) of the foamable carrier or composition. Preferably, the propellant is from about 5% to about 12%. Examples of suitable propellants include volatile hydrocarbons such as butane, propane, isobutane or mixtures thereof, and fluorocarbon gases. As can be noted from the examples the added propellant is usually expressed as a percentage of the total composition such that all the other ingredients combine to be 100% and the propellant is added on to the 100%.

Aerosol propellants are used to generate and administer the foamable composition as a foam. Suitable propellants include volatile hydrocarbons such as butane, propane, isobutane and fluorocarbon gases, or mixtures thereof.

In an embodiment the propellant is 1681 a mixture of propane, isobutene and butane. In an embodiment the propellant is AP 70 which is another mixture of propane, isobutene and butane. In another embodiment the propellant is AP 46 which is a similar mixture of propane, isobutene and butane but having a lower pressure. AP 70 offers about 50% higher pressure than AP 46.

In some circumstances the propellant may be up to 35%. The propellants are used to generate and administer the foamable composition as a foam. The total composition including propellant, foamable compositions and optional ingredients can be referred to as the foamable composition.

Alcohol and organic solvents render foams inflammable. It has been surprisingly discovered that fluorohydrocarbon propellants, other than chloro-fluoro carbons (CMOs), which are non-ozone-depleting propellants, are particularly useful in the production of a non-flammable foamable composition. A test according to European Standard prEN 14851, titled “Aerosol containers—Aerosol foam flammability test” revealed that compositions containing an organic carrier that contains a hydrophobic organic carrier and/or a polar solvent, which are detected as inflammable when a hydrocarbon propellant is used, become non-flammable, while the propellant is an HFC propellant.

Such propellants include, but are not limited to, hydrofluorocarbon (HFC) propellants, which contain no chlorine atoms, and as such, fall completely outside concerns about stratospheric ozone destruction by chlorofluorocarbons or other chlorinated hydrocarbons. Exemplary non-flammable propellants according to this aspect include propellants made by DuPont under the registered trademark Dymel, such as 1,1,1,2 tetrafluorethane (Dymel 134), and 1,1,1,2,3,3,3 heptafluoropropane (Dymel 227). HFCs possess Ozone Depletion Potential of 0.00 and thus, they are allowed for use as propellant in aerosol products.

Notably, the stability of foamable emulsions including HFC as the propellant can be improved in comparison with the same composition made with a hydrocarbon propellant.

In one or more embodiments foamable compositions comprise a combination of a HFC and a hydrocarbon propellant such as n-butane or mixtures of hydrocarbom propellants such as propane, isobutane and butane.

### Aging

In order to project the potential shelf life and stability of the compositions and their ingredients particularly active or benefit agents the compositions can subjected to a number of tests, including centrifugation to look for resistance to creaming, phase separation; one or more freeze thaw cycles, standing at room and higher temperatures as an indicator of resistance to aging.

### Substantially Alcohol-Free

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butaneol, iso-butaneol, t-butaneol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

### Shakability

‘Shakability’ means that the composition contains some or sufficient flow to allow the composition to be mixed or remixed on shaking. That is, it has fluid or semi fluid properties. In some very limited cases it may exceptionally be possible to have a foamable composition which is flowable but not apparently shakable.

### Breakability

A breakable foam is one that is thermally stable, yet breaks under sheer force.

The breakable foam of the present invention is not “quick breaking”, i.e., it does not readily collapse upon exposure to body temperature environment. Sheer-force breakability of the foam is clearly advantageous over thermally induced breakability, since it allows comfortable application and well directed administration to the target area.

### Active Agents

It is to be understood that the active agents useful herein can in some instances provide more than one benefit or operate via more than one mode of action. Therefore, classifications herein are made for the sake of convenience and are not intended to limit the active agent to that particular application or applications listed.

The composition of the present invention comprises an active agent that provides therapeutic or cosmetic activity.

Non-limiting examples of active agents include an anti-infective, an antibiotic, an antibacterial agent, an antifungal agent, an antiviral agent, an antiparasitic agent, a steroidal anti-inflammatory agent, a nonsteroidal anti-inflammatory agent, an immunosuppressive agent, an immunomodulator, an immunoregulating agent, a hormonal agent, a steroid, a vasoactive agent, a vasoconstrictor, a vasodilator, vitamin A, a vitamin A derivative, a retinoid, vitamin B, a vitamin B derivative, vitamin C, a vitamin C derivative, vitamin D, a vitamin D derivative, vitamin E, a vitamin E derivative, alpha-tocopheryl polyethylene glycol succinate, vitamin F, a vitamin F derivative, vitamin K, a vitamin K derivative, a wound healing agent, a burn healing agent, a disinfectant, an anesthetic, an antiallergic agent, an alpha hydroxyl acid, lactic acid, glycolic acid, a beta-hydroxy acid, a protein, a peptide, a neuropeptide, an allergen, an immunogenic substance, a haptene, an oxidizing agent, an antioxidant, a dicarboxylic acid, azelaic acid, sebacic acid, adipic acid, fumaric acid, an insecticide, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide), silicone oxide, talc, an anti-acne agent, a skin whitening agent, a self tanning agent, an anti-cellulite agent, a skin protective agent, a masking agent, an anti-wart agent, a refatting agent, a lubricating agent and mixtures thereof at any proportion. The concentration of the active agent can be adapted to exert a therapeutic effect on a disease when applied to an afflicted area.

In one or more embodiments the active agent may be an extract or tincture of one or more beneficial agents that have beneficial properties, for example, when applied to the skin, a body surface, a body cavity or a mucosal surface. The extract can be, for example, alcoholic, hydroalcoholic, propelyne glycol, glycerine, dry, press, cold, hot, liquid carbon dioxide, oil or other process known in the art. The extract or tincture may comprise of substances of animal, plant, (such as herb, fruit, vegetable) mineral or other origin. Nonlimiting examples are proteins, polypepeptides, sugars, hyularonic acid, and coal tar. Herbal extracts may be from any known therapeutic herb, as listed for example in Herbal Medicines, London: Pharmaceutical Press Electronic Version 2006 or in the American Herbal Association electronic publication Herbal gram or in German Commission E., such as, angelica, calendula, celery, coltsfoot, comfrey, dandelion, jamaica dogwood, kava, marshmallow, prickly ash, northern prickly ash, southern senna, valerian, agrimony, aloe vera, alfalfa, artichoke, avens, bayberry, bloodroot, blue flag, bogbean, boldo, boneset, broom, buchu, burdock, burnet, calamus, calendula, cascara, centaury, cereus, chamomile, german chamomile, roman chamomile, cinnamon, clivers, cohosh, black, cohosh, blue, cola, corn silk, couchgrass, cowslip, damiana, devil's claw, drosera, echinacea, elder, elecampane, euphorbia, eyebright, figwort, frangula, fucus, fumitory, garlic, golden seal, gravel root, ground ivy, guaiacum, hawthorn, holy thistle, hops, horehound black, horehound white, horse chestnut hydrangea, ispaghula, juniper, lady's lipper, liferoot, lime flower, liquorice, lobelia, mate, meadowsweet, mistletoe, motherwort, myrrh, nettle, parsley, parsley piert, passionflower, pennyroyal, pilewort, plantain, pleurisy root, pokeroot, poplar, pulsatilla, queen's delight, raspberry, red clover, rosemary, sage, sarsaparilla, sassafras, scullcap, senega, shepherd's purse, skunk cabbage, slippery elm, squill, St. John's wort, stone root, tansy, thyme, uva-ursi, vervain, wild carrot, wild lettuce, willow, witch hazel, yarrow and yellow dock. The extract may contain, for example, an aqueous, polar, hydrophobic or potent solvent as will be appreciated by a person in the art.

In one or more embodiments, the active agent is an anti-infective agent, selected from an antibiotic agent, an antibacterial agent, an anti-fungal agent, an anti-viral agent and an anti-parasite agent.

The antibacterial drug can be active against gram positive and gram-negative bacteria, protozoa, aerobic bacteria and anaerobic ones.

In one or more embodiments, the antibiotic agent is selected from the classes consisting of beta-lactam antibiotics, synthetic and semi-synthetic penicillin's, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, fluoroquinolnes, antibiotic steroids, cyclosporines, sulfonamides, tetracycline, chloramphenicol, dicarboxylic acids, such as azelaic acid, salicylates, antibiotic metals, oxidizing agents, substances that release free radicals and/or active oxygen, cationic antimicrobial agents, quaternary ammonium compounds, biguanides, triguanides, bisbiguanides and analogs and polymers thereof and naturally occurring antibiotic compounds.

Additional antibacterial agents, which are non-specific, include strong oxidants and free radical liberating compounds, such as hydrogen peroxide, bleaching agents (e.g., sodium, calcium or magnesium hypochloride and the like), iodine, chlorohexidine and benzoyl peroxide.

The antifungal agent can be an azole compound. Exemplary azole compounds include azoles selected from the group consisting of azoles, diazoles, triazoles, miconazole, ketoconazole, clotrimazole, econazole, mebendazole, bifonazole, butoconazole, fenticonazole, isoconazole, oxiconazole, sertaconazole, sulconazole, thiabendazole, tiaconazole, fluconazole, itraconazole, ravuconazole and posaconazole.

Additional exemplary antifungal agents include griseofulvin, ciclopirox, amorolfine, terbinafine, Amphotericin B, potassium iodide, flucytosine (5FC) and any combination thereof at a therapeutically effective concentration.

In one or more embodiments, the active agent is an anti-viral agent. Any known antiviral agent, in a therapeutically effective concentration, can be incorporated in the foam composition of the present invention. Exemplary antiviral agents include, but not limited to, acyclovir, famciclovir, gancyclovir, valganciclovir and abacavir.

In another embodiment according to the present invention, the active agent is an anti-inflammatory or anti-allergic agent. Anti-inflammatory agents can be selected from the group of corticosteroids, non-steroidal anti-inflammatory drugs (NSAIDs), anti-histamines, immunosuppressant agents, immunomodulators; and any combination thereof at a therapeutically effective concentration.

Non-limiting examples of corticosteroids include hydrocortisone, hydrocortisone acetate, desonide, betamethasone valerate, clobetasone-17-butyrate, flucinonide, fluocinolone acetonide, alcometasone dipropionate, mometasone furoate, prednicarbate, triamcinolone acetonide, betamethasone-17-benzoate, methylprednisolone aceponate, betamethasone dipropionate, halcinonide, triamcinolone acetonide, halobetasol and clobetasol-17-propionate.

A second class of anti-inflammatory agents, which is useful in the foam of the present invention, includes the nonsteroidal anti-inflammatory agents (NSAIDs). The variety of compounds encompassed by this group is well known to those skilled in the art. Specific non-steroidal anti-inflammatory agents useful in the composition invention include, but are not limited to, oxicams, such as piroxicam, isoxicam, tenoxicam, sudoxicam; salicylates, such as salicylic acid, ethyl salicylate, methyl salycilate, aspirin, disalcid, benorylate, trilisate, safapryn, solprin, diflunisal, and fendosal; scetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and pyrazoles, such as phenylbutazone, oxyphenbutazone, feprazone, azapropazone, and trimethazone.

Any further steroidal and nonsteroidal compounds, having the capacity to prevent, alleviate the symptoms of, treat or cure inflammation processes, are generally included, as possible anti-inflammatory agents, according to the present invention.

Antiallergic active agents include antihistamine compounds, including, in a non limiting manner, thylenediamines, such as pyrilamine (mepyramine), antazoline and methapyrilene; tripelennamine phenothiazines, such as promethazine, methdilazine and trimeprazine; ethanolamines, such as diphenhydramine, bromodiphenhydramine, carbinoxamine, clemastine, dimenhydrinate, diphenylpyraline, doxylamine and phenyltoxamine; piperazines, such as cyclizine, buclizine, chlorcyclizine, hydroxyzine, meclizine and thiethylperazine; alkylamines, such as brompheniramine, pyrrobutamin, desbrompheniramine, tripolidine, dexchlorpherniramine, chlorpheniramine; dimethindene and pheniramine; and piperidines, such as cyproheptadine and azatadine. These active agents, as well as additional antihistamines can also be incorporated in the composition of the present invention.

The composition of the present invention may also comprise an anti-inflammatory or antiallergic agent, wherein said agent reduces the occurrence of pro-inflammatory cytokines or inhibits the effect of pro-inflammatory cytokines.

Immunosuppressant agents, immunoregulating agents and immunomodulators are chemically or biologically derived agents that modify the immune response or the functioning of the immune system (as by the stimulation of antibody formation or the inhibition of white blood cell activity). Immunosuppressant agents and immunomodulators include, among other options, cyclic peptides, such as cyclosporine, tacrolimus, tresperimus, pimecrolimus, sirolimus (rapamycin), verolimus, laflunimus, laquinimod and imiquimod.

In one or more embodiments, the active agent is a topical anesthetic. Examples of topical anesthetic drugs include, but not limited to, benzocaine, lidocaine, bupivacaine, chlorprocaine, dibucaine, etidocaine, mepivacaine, tetracaine, dyclonine, hexylcaine, procaine, cocaine, ketamine, pramoxine, and phenol. Mixtures of such anesthetic agents may be synergistically beneficial.

In one or more embodiments, the active agent is a “keratolytically active agent.” The term “keratolytically active agent” refers herein to a compound, which loosens and removes the stratum corneum of the skin, or alters the structure of the keratin layers of the skin.

Suitable keratolytically active agents include phenol and substituted phenolic compounds. Such compounds are known to dissolve and loosen the intracellular matrix of the hyperkeratinized tissue. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic.

Vitamin A and its derivatives, such as retinoic acid, isoretinoic acid, retinol and retinal are another preferred class of keratolytically active agents.

Another group of keratolytically active agents include alpha-hydroxy acids, such as lactic acid and glycolic acid and their respective salts and derivatives; and beta-hydroxy acids, such as Salicylic acid (o-hydroxybenzoic acid) and its salts and pharmaceutically acceptable derivatives, which typically possess anti-inflammatory, as well as keratolytic, activity. Yet, another class of preferred keratolytically active agents includes urea and its derivatives.

In one or more embodiments, the active agent is a retinoid. Retinoids include, for example, retinol, retinal, all-trans retinoic acid and derivatives, isomers and analogs thereof. Etretinate, actiretin, isotretinoin, adapalene and tazarotene are further examples of said retinoid isomers and analogs.

In one or more embodiments, the active agent is an insecticide or an insect repellent agent.

In one or more embodiments, the active agent is an anti cancer agent.

In one or more embodiments, the active agent is a photodynamic therapy (PDT) agent. By way of example, such PDT agents can be modified porphyrins, chlorins, bacteriochlorins, phthalocyanines, naphthalocyanines, pheophorbides, purpurins, m-THPC, mono-L-aspartyl chlorin e6, bacteriochlorins, phthalocyanines, benzoporphyrin derivatives, as well as photosensitizer precursors, such as aminolevulinic acid (ALA).

In one or more embodiments, the active agent is an agent useful in the treatment of burns, wounds, cuts and ulcers. The foam compositions of the present invention may comprise a combination of anti-infective agents (against bacteria, fungi and/or viruses), anti-inflammatory agents (steroidal and/or NSAIDs) and pain relieving components.

In one or more embodiments, the active agent can also be used as an absorption and bioavailability enhancer for other drugs and vitamins, for example TPGS that forms its own micelles can aid e.g. amprenavir and vitamin D respectively.

In one or more embodiments the active agent has some degree of solubility in water. By the phrase some degree of solubility it is understood to include API's that are described by the US or European Pharmacopoeia as being slightly soluble, sparingly soluble, soluble, freely soluble or very soluble. Both describe the approximate ranges of parts of solvent (volume) required for 1 part (per gram) of solute as less than 1 for very soluble; from 1-10; for freely soluble, from 10-30 for soluble; from 30 to 100 for sparingly soluble; and from 100 to 1000 for slightly soluble. Additionally, the phrase may include the terms partly soluble and miscible. Non limiting examples of substances that have some degree of solubility in water are acyclovir, azelaic acid, allantoin, ammonium lactate, benzoyl peroxide, caffeine, calcipotriol, ciclopirox olamine, clindamycin hydrochloride, clindamycin phosphate, clindamycin palmitate hydrochloride, coal tar, cyanocobalamine, diclofenac sodium, gentamycin sulphate, lactic acid, glycyrrhizinic acid, map (magnesium ascorbyl phosphate), minoxidil, mupirocin, salicylic acid, terbinafine, urea, fusidic acid, hydrocortisone sodium phosphate, hydrocortisone sodium succinate, ketoconazole, lidocaine hydrochloride, metronidazole, tetracycline, tetracycline hydrochloride, meclocycline sulfosalicylate, resorcinol, chloramphenicol, erythromycin, acriflavinium monochloride, ethacridine lactate, dibrompropamidine isetionate, chlorhexidine acetate, chlorhexidine gluconate, chlorhexidine hydrochloride, hexamidine isetionate, phenol, povidone-iodine, dequalinium chloride, hydroxyquinoline sulfate, potassium hydroxyquinoline sulphate, benzalkonium chloride, cetrimonium bromide, cetylpyridinium chloride, cetrimide, phenylmercuric acetate, phenylmercuric borate, mercuric chloride, silver nitrate, potassium permanganate, tosylchloramide sodium, prednisolone sodium phosphate, betamethasone sodium phosphate, demeclocycline, demeclocycline hydrochloride, chlortetracycline hydrochloride, oxytetracycline hydrochloride, neomycin sulfate, bacitracin zinc, gentamicin sulphate, amikacin, amikacin sulphate, sulfathiazole sodium, mafenide acetate, idoxuridine, fumaric acid, mepyramine maleate, tripelennamine hydrochloride, promethazine hydrochloride, dimetindene maleate, diphenhydramine hydrochloride, cinchocaine hydrochloride, oxybuprocaine hydrochloride, benzocaine, tetracaine hydrochloride, pramoxine hydrochloride, panthenol, dexpanthenol, calcium pantothenate, hyaluronic acid, trypsin, aminobenzoic acid, methylrosanilinium chloride, sodium butyl hydroxybenzoate, sodium ethyl hydroxybenzoate, sodium methyl hydroxybenzoate, sodium propyl hydroxybenzoate, flucytosine and fluconazole.

In one or more embodiments the active agent has a limited degree of solubility in water. By a limited degree of solubility it is understood to include API's that are described by the US or European Pharmacopoeia as being very slightly soluble. The approximate range of parts of solvent (volume) required for 1 part (per gram) of solute is from 1000 to 10000 for very slightly soluble.

In one or more embodiments the active agent has some degree of solubility in an unctuous emollient. So any agent that by its nature is hydrophobic may qualify, such as permethrin and tetracaine.

In one or more embodiments the active agent has some degree of solubility in a composition of the present invention in one or more of the water phase, the oil phase, or the interphase or the foam. For example, beamethasone valerate has been stated to be practically insoluble in water. However, it has been surprisingly found that it is soluble in the water phase of a foamable composition in a pharmaceutically effective amount for topical application.

The foam compositions of the present invention, with or without further active ingredients, are suitable for the further application as “cosmeceutical” preparation (cosmetic products with therapeutic benefit), to treat “cosmetic” skin disorders, such as aging skin, wrinkles, hyperpigmentation (melasma, chloasma, freckles, etc.), scaly skin and other skin undesirable properties.

Any cosmetically active agent is considered an active agent in the context of the present invention. The CTFA Cosmetic Ingredient Handbook describes a wide variety of non-limiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, astringents, etc. (e.g., clove oil, menthol, camphor, eucalyptus oil, eugenol, menthyl lactate, witch hazel distillate), anti-acne agents, anti-caking agents, antifoaming agents, anti-microbial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, moisturizers, etc.), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, and vitamins and derivatives thereof.

In one or more embodiments, the active agent is an agent useful in the treatment of acne, wrinkles and scars. Examples of useful anti-acne actives include resorcinol, sulfur, salicylic acid and salicylates, alpha-hydroxy acids, nonsteroidal anti-inflammatory agents, benzoyl peroxide, retinoic acid, isoretinoic acid and other retinoid compounds, adapalene, tazarotene, azelaic acid and azelaic acid derivatives, antibiotic agents, such as erythromycin and clyndamycin, zinc salts and complexes, and combinations thereof, in a therapeutically effective concentration. Exemplary anti-wrinkle/anti-atrophy active agents suitable for use in the compositions of the present invention include sulfur-containing D and L amino acids and their derivatives and salts, particularly the N-acetyl derivatives; thiols; hydroxy acids (e.g., alpha-hydroxy acids such as lactic acid and glycolic acid and their derivatives and salts; or beta-hydroxy acids such as salicylic acid and salicylic acid salts and derivatives), urea, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate). In the case of dry, scaly skin (xerosis) and ichthyosis such agents can alleviate the symptoms by temporary relief of itching associated with these conditions.

In one or more embodiments, the active agent is an anti-oxidant or a radical scavenger. Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid, gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used.

It is further pointed out that polyunsaturated fatty acids, containing omega-3 and omega-6 fatty acids (e.g., linoleic and linolenic acid, gamma-linoleic acid (GLA), eicosapentaenoic acid (EPA) and docosahexaenoic acid (DHA)) are beneficial in the treatment of psoriasis and other skin inflammation conditions. Likewise, emollients and silicone oils exert moisture-retaining and skin protective effects on the skin. Thus, in a preferred embodiment, a skin protective foam is provided, wherein the hydrophobic carrier comprises in full or in part, an organic liquid selected from the group consisting of emollients, silicone oil and oils rich in unsaturated fatty acids.

In one or more embodiments, the active agent is a self-tanning active Agent, such as dihydroxyacetone.

According to another embodiment, the active agent comprises solid matter or particulate matter, i.e., material that is not soluble in the liquid carrier composition of the foamable composition. For definition purposes, solid matter shall mean material that is not soluble in the foamable composition more than 10% of the concentration intended to be included in said foamable composition. By way of example, the following classes of solid matter substances are presented: metallic oxides, such as titanium dioxide, zinc oxide, zirconium oxide, iron oxide; silicon containing materials such as silicone oxide and talc; carbon, for example in the form of amorphous carbon or graphite; insoluble oxidizing agents, such as benzoyl peroxide, calcium and magnesium hypochlorite; metallic Silver; cosmetic scrub materials, including, for example meals of strawberry seeds, raspberry seeds, apricot seeds, sweet almond, cranberry seeds; and pigments. In an embodiment of the present invention the solid is substantially uniformly dispersed as a suspension in the composition, wherein the composition is formulated so that the resultant foam when applied topically to a target will nor per se form an effective occlusive barrier, is not completely occlusive; is not sufficient to form an occlusive barrier or any occlusiveness is significantly transient; or so that the composition does not comprise an organic cosolvent.

According to certain embodiments, the active agent is selected from the group of solvent, surface active agent, foam adjuvant and gelling agent, which are, on a case-by-case basis, known to possess a therapeutic benefit.

In one or more embodiments at least one or at least two active agents are included in the composition.

### Composition and Foam Physical Characteristics and Advantages

A pharmaceutical or cosmetic composition manufactured using the foamable carrier of the present invention is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foamable composition can be in the state of (1) solutions; (2) a readily dispersible suspension; or (3) an emulsion. It is stable, having an acceptable shelf life of a year, or at least two years at ambient temperature, as revealed in accelerated stability tests. Polar solvents, hydrophobic carriers and propellants, which are a mixture of low molecular weight hydrocarbons, tend to impair the stability of emulsions and to interfere with the formation of a stable foam upon release from a pressurized container. It has been observed, however, that the foamable compositions according to the present invention are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam. Compositions containing semi-solid hydrophobic solvents, e.g., white petrolatum, as the main ingredients of the oil phase of the emulsion, exhibit high viscosity and reduced or poor flowability and are not ideal candidates for a foamable composition. It has been found that despite the aforesaid in the compositions of the present inventions the produce foams, which are surprisingly soft, or with improved stability.

Where the unctuous emollient is provided in large quantities sufficient to produce an effective occlusion the foam can act as a barrier to water soluble irritants and air borne bacteria whilst also providing a vehicle for water soluble active agents. However, there is a potential downside of anaerobic bacteria growing under the barrier. Depending on the nature of the emulsion formulation an unctuous emollient can aid API transport through the skin or retard penetration prolonging thereby its action. Accordingly a pharmaceutical formulation for example with petrolatum can be designed to improve or prolong delivery as is required as will be appreciated by a person skilled in the art.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

As a further aspect of the foam is breakability. The breakable foam is thermally stable, yet breaks under sheer force. Sheer-force breakability of the foam is clearly advantageous over thermally induced breakability. Thermally sensitive foams immediately collapse upon exposure to skin temperature and, therefore, cannot be applied on the hand and afterwards delivered to the afflicted area.

The foam of the present invention has several advantages, when compared with hydroalcoholic foam compositions. The foam of the present invention is thermally stable. Unlike hydroalcoholic foam compositions of the prior art, the foam of the present invention is not “quick breaking”, i.e., it does not readily collapse upon exposure to body temperature environment. Sheer-force breakability of the foam is clearly advantageous over thermally induced breakability, since it allows comfortable application and well directed administration to the target area;
(1) Skin drying and skin barrier function. Polar solvents and or potent solvents can dry the skin and impair the integrity of the skin barrier. By contrast, combining a polar solvent and or potent solvent with an unctuous emollient and or a hydrophobic carrier, as described herein, unwanted skin barrier damage is reduced, as can be demonstrated in trans-epidermal water loss measurements; and
(2) Irritability. Due to the improvement in skin barrier function, or further through addition of a humectants or a moisturizer skin irritability is corrected or ameliorated.

In terms of usability, the foamable composition is most advantageous, as revealed by clinical trials:

(i) Ease of Application.
When foam is released it expands and allows easy spreading on the target area. This advantage is particularly meaningful in regards to the treatment of large skin surfaces.
Upon application, the foam readily spreads and absorbs into the skin.

(ii) The Foam is Drip-Free
The foam is not liquid and therefore does not leak when applied.
This allows precise application, without the product being spread on clothes or other parts of the body.

For the purpose of the specification the external limits of the various ranges given are approximate as will be appreciated by those skilled in the art. Therefore, for the purpose of interpreting the outer limits of the range the limits shall be deemed to include up to a 20% leeway outside the range, preferably a 10% leeway.

### Fields of Applications

According to one or more embodiments of the present invention, the foamable carrier and the foamable pharmaceutical or cosmetic composition of the present invention are intended for administration to an animal or a human subject. In one or more embodiments, the composition is intended to treat the skin, a body surface, a body cavity or a mucosal surface, e.g., the mucosa of the nose, mouth, eye, ear, respiratory system, vagina or rectum.

By including an appropriate active agent in the compositions of the present invention, the composition are useful in treating a patient having any one of a variety of dermatological disorders, which include inflammation as one or their etiological factors (also termed “dermatoses”), such as classified in a non-limiting exemplary manner according to the following groups:

Dermatitis including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash; Bacterial infections including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, and erythrasma;

Fungal Infections including dermatophyte infections, yeast Infections; parasitic Infections including scabies, pediculosis, creeping eruption;

Viral Infections;

Disorders of hair follicles and sebaceous glands including acne, rosacea, perioral dermatitis, hypertrichosis (hirsutism), alopecia, including male pattern baldness, alopecia greata, alopecia universalis and alopecia totalis; pseudofolliculitis barbae, keratinous cyst;

Scaling papular diseases including psoriasis, pityriasis rosea, lichen planus, pityriasis rubra pilaris;

Benign tumors including moles, dysplastic nevi, skin tags, lipomas, angiomas, pyogenic granuloma, seborrheic keratoses, dermatofibroma, keratoacanthoma, keloid;

Malignant tumors including basal cell carcinoma, squamous cell carcinoma, malignant melanoma, paget's disease of the nipples, kaposi's sarcoma;

Reactions to sunlight, including sunburn, chronic effects of sunlight, photosensitivity;

Bullous diseases including pemphigus, bullous pemphigoid, dermatitis herpetiformis, linear immunoglobulin A disease;

Pigmentation disorders including hypopigmentation such as vitiligo, albinism and postinflammatory hypopigmentation and hyperpigmentation such as melasma (chloasma), drug-induced hyperpigmentation, postinflammatory hyperpigmentation;

Disorders of cornification including ichthyosis, keratosis pilaris, calluses and corns, actinic keratosis;

Pressure sores, open wounds, chronic wounds, open ulcers and burns;

Disorders of sweating; and

Inflammatory reactions including drug eruptions, toxic epidermal necrolysis, erythema multiforme, erythema nodosum, and granuloma annulare.

The same advantage is expected when the composition is topically applied to a body cavity or mucosal surfaces, including, but not limited to the cranial cavity, the thoratic cavity, the abdominal cavity, the venteral cavity, the vagina, the rectum and penile cavities, the urinary tract, the nasal cavity, the mouth, the eye, the ear the peritoneum, the large and small bowel, the caecum, bladder, and stomach, the cavity between the uterus and the fallopian tubes, the ovaries and other body areas, which may accept topically-applied products. The composition of the present invention is suitable to treat conditions of a body cavity and a mucosal membrane, such as post-surgical adhesions, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

According to one or more embodiments of the present invention, the compositions are also useful in the therapy of non-dermatological disorders by providing transdermal or trans-mucosal delivery of an active agent that is effective against non-dermatological disorders.

In one or more embodiments, the disorder is a health abnormality that responds to treatment with a hormone. A typical example of such abnormality is sexual dysfunction in men and women whereby androgen therapy is successfully used to restore sexual function. Other non-limiting examples of disorders/medical indications that are in the scope of treatment with a hormone according to the present invention are androgen deficiency, estrogen deficiency, growth disorders, hypogonadism, cancer, vasomotor symptoms, menopausal disorders, vulvar and vaginal atrophy, urethritis, hypoestrogenism, osteoarthritis, osteoporosis, uterine bleeding, Hirsutism, Virilization, ovarian tumors, hypothalamic pituitary unit diseases, testicular tumors, prostate cancer, hypopituitarism, Klinefelter's syndrome, testicular feminisation, orchitectomy, vasomotor symptoms (such as “hot flashes”) associated with the menopause, metabolic abnormalities and mood disturbances.

Other foamable compositions are described in: U.S. Publication No. 05-0232869, published on Oct. 20, 2005, entitled NONSTEROIDAL IMMUNOMODULATING KIT AND COMPOSITION AND USES THEREOF; U.S. Publication No. 05-0205086, published on Sep. 22, 2005, entitled RETINOID IMMUNOMODULATING KIT AND COMPOSITION AND USES THEREOF; U.S. Publication No. 06-0018937, published on Jan. 26, 2006, entitled STEROID KIT AND FOAMABLE COMPOSITION AND USES THEREOF; U.S. Publication No. 05-0271596, published on Dec. 8, 2005, entitled VASOACTIVE KIT AND COMPOSITION AND USES THEREOF; U.S. Publication No. 06-0269485, published on Nov. 30, 2006, entitled ANTIBIOTIC KIT AND COMPOSITION AND USES THEREOF; U.S. Publication No. 07-0020304, published on Jan. 25, 2007, entitled NON-FLAMMABLE INSECTICIDE COMPOSITION AND USES THEREOF; U.S. Publication No. 06-0193789, published on Aug. 31, 2006, entitled FILM FORMING FOAMABLE COMPOSITION; U.S. patent application Ser. No. 11/732,547, filed on Apr. 4, 2007, entitled ANTI-INFECTION AUGMENTATION OF FOAMABLE COMPOSITIONS AND KIT AND USES THEREOF; U.S. Provisional Patent Application No. 60/789,186, filed on Apr. 4, 2006, KERATOLYTIC ANTIFUNGAL FOAM; U.S. Provisional Patent Application No. 0/815948, filed on Jun. 23, 2006, entitled FOAMABLE COMPOSITIONS COMPRISING A CALCIUM CHANNEL BLOCKER, A CHOLINERGIC AGENT AND A NITRIC OXIDE DONOR; U.S. Provisional Patent Application No. 60/818,634, filed on Jul. 5, 2006, entitled DICARBOXYLIC ACID FOAMABLE VEHICLE AND PHARMACEUTICAL COMPOSITIONS THEREOF; U.S. Provisional Patent Application No. 60/843,140, filed on Sep. 8, 2006, entitled FOAMABLE VEHICLE AND VITAMIN PHARMACEUTICAL COMPOSITIONS THEREOF, all of which are incorporated herein by reference in their entirety with reference to any of the active ingredients; excipients; surfactants; penetration enhancers; humectants; moisturizers; listed therein can be applied herein and are incorporated by reference.

The following examples further exemplify the benefit agent foamable pharmaceutical carriers, pharmaceutical compositions thereof, methods for preparing the same, and therapeutic uses of the compositions. The examples are for the purposes of illustration only and are not intended to be limiting. Many variations may be carried out by one of ordinary skill in the art and are contemplated within the full scope of the present invention.

Methodology

A general procedure for preparing foamable compositions is set out in WO 2004/037225, which is incorporated herein by reference.

Emulsion Foam
1. Mix oily phase ingredients and heat to 75° C. to melt all ingredients and obtain homogeneous mixture.
2. Mix polymers in water with heating or cooling as appropriate for specific polymer. Whilst the polymers may be added instead into the oily phase it was found to be advantageous to prepare them in the water phase.
3. Add all other water soluble ingredients to water-polymer solution and heat to 75° C.
4. Add slowly internal phase to external phase at 75° C. under vigorous mixing and homogenize to obtain fine emulsion. Alternatively the external phase is added slowly to the internal phase.
5. Cool to below 40° C. and add sensitive ingredients with mild mixing.
6. Cool to room temperature.

Oily Foam with Phospholipids and/or Water
b 1. Swell the phospholipids in the main oily solvent under mixing for at least 20 minutes until uniform suspension is obtained.
2. Add all other ingredients excluding polymers and heat to 75° C. to melt and dissolve and obtain homogeneous mixture.
3. Mix well and cool to below 40° C. and add the polymers and sensitive ingredients with moderate mixing.
4. Cool to room temperature.
5. In case of polymers dissolved in water or organic solvent, dissolve the polymers in the solvent with heating or cooling as appropriate for specific polymer and add to the oily mixture under vigorous mixing at ˜40° C.

SCanisters Filling and Crimping

Each aerosol canister is filled with PFF and crimped with valve using vacuum crimping machine.

### Pressurizing

### Propellant Filling

Pressurizing is carried out using a hydrocarbon gas or gas mixture

Canisters are filled and then warmed for 30 sec in a warm bath at 50° C. and well shaken immediately thereafter.

### Closure Integrity Test.

Each pressurized canister is subjected to bubble and crimping integrity testing by immersing the canister in a 60° C. water bath for 2 minutes. Canisters are observed for leakage as determined by the generation of bubbles. Canisters releasing bubbles are rejected.

Tests

By way of non limiting example the objectives of hardness, collapse time, viscosity, bubble size, nano size and FTC stability tests are briefly set out below as would be appreciated by a person of the art.

Hardness

LFRA100 instrument is used to characterize hardness. A probe is inserted into the test material. The resistance of the material to compression is measured by a calibrated load cell and reported in units of grams on the texture analyzer instrument display. Preferably at least three repeat tests are made. The textural characteristics of a dispensed foam can effect the degree of dermal penetration, efficacy, spreadability and acceptability to the user. The results can also be looked at as an indicator of softness. Note: the foam sample is dispensed into an aluminum sample holder and filled to the top of the holder.

Collapse Time

Collapse time (CT) is examined by dispensing a given quantity of foam and photographing sequentially its appearance with time during incubation at 36° C. It is useful for evaluating foam products, which maintain structural stability at skin temperature for at least 1 min.

Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000CP, the viscosity at 1 RPM may be measured, although the figures are of a higher magnitude. Unless otherwise stated viscosity of the pre foam formulation is provided.

FTC (Freeze Thaw Cycles)

To check the foam appearance under extreme conditions of repeated cycles of cooling, heating, (first cycle) cooling, heating (second cycle) etc., commencing with −10° C. (24 hours) followed by +40° C. (24 hours) measuring the appearance and again repeating the cycle for up to four times.

Creaming by Centrifugation:

1. Principle of Test

The centrifugation used in this procedure serves as a stress condition simulating the aging of the liquid dispersion under investigation. Under these conditions, the centrifugal force applied facilitates the coalescence of dispersed globules or sedimentation of dispersed solids, resulting in loss of the desired properties of the formulated dispersion.

2. Procedure
2.1. Following preparation of the experimental formulation/s, allow to stand at room temperature for ≧24 h.
2.2. Handle pentane in the chemical hood. Add to each experimental formulation in a 20-mL glass vial a quantity of pentane equivalent to the specified quantity of propellant for that formulation, mix and allow formulation to stand for at least 1 h and not more than 24 h.
2.3. Transfer each mixture to 1.5 mL microtubes. Tap each microtube on the table surface to remove entrapped air bubbles.
2.4. Place visually balanced microtubes in the centrifuge rotor and operate the centrifuge at one or more of 10,000 rpm for 10 min, 3,000 rpm for 10 min or at 1,000 rpm for 10 min.

Bubble size:

Foams are made of gas bubbles entrapped in liquid. The bubble size and distribution reflects in the visual texture and smoothness of the foam. Foam bubbles size is determined by dispensing a foam sample on a glass slide, taking a picture of the foam surface with a digital camera equipped with a macro lens. The diameter of about 30 bubbles is measured manually relatively to calibration standard template. Statistical parameters such as mean bubble diameter, standard deviation and quartiles are then determined. Measuring diameter may also be undertaken with image analysis software. The camera used was a Nikon D40X Camera (resolution 10MP) equipped with Sigma Macro Lens (ref: APO MACRO 150 mm F2.8 EX DG HSM). Pictures obtained are cropped to keep a squared region of 400 pixels×400 pixels.

Microscope Size:

The light microscope enables observing and measuring particles from few millimeters down to one micron. Light microscope is limited by the visible light wavelength and therefore is useful to measuring size of particles above 800 nanometers and practically from 1 micron (1,000 nanometers).

### Stock Compositions

Non-limiting examples of how stock solutions are made up with and without API. Other stock solutions may be made using the same methodology by simply varying adding or omitting ingredients as would be appreciated by a man of the art.

### EXAMPLES

### Part A

### Example 1

### Foamable Carrier with 12.5% and 25% Petrolatum as Unctuous Emollient and Steareth-2/Steareth-20/ASOS as Multi-Active Component

| Phase | Ingredient name: (INCI, CTFA) | | % w/w | % w/w |
|---|---|---|---|---|
| Water | Water | | 76.05 | 63.55 |
| phase (A) | Carbomer 941 | | 0.12 | 0.12 |
| | sodium Lauryl sulfate | | 0.10 | 0.10 |
| | Disodium EDTA | | 0.05 | 0.05 |
| | Methylparaben | | 0.20 | 0.20 |
| Oil phase | Petrolatum | | 12.50 | 25.00 |
| (B) | Stearath-2 | | 1.73 | 1.73 |
| | Steareth-20 | | 0.58 | 0.58 |
| | Propylparaben | | 0.15 | 0.15 |
| | Cetyl alcohol | | 0.10 | 0.10 |
| | Cyclomethicone | | 5.00 | 5.00 |
| | ASOS | | 3.00 | 3.00 |
| | Imidazolidinyl urea | | 0.30 | 0.30 |
| | Triethanolamine | | 0.12 | 0.12 |
| | Total | | 100.00 | 100.00 |
| | Propellant (Propane/Butane/ | | 8.00 | 8.00 |
| | Isobutane) | | | |
| Results | Appearance | Foam quality | E | E |
| | | Color | W | W |
| | | Odor | No odor | No odor |

| | | |
|---|---|---|
| Shaking ability | Good | Moderate |
| Density | 0.140 | 0.159 |
| Hardness | 14.96 | 15.99 |
| Collapse time at 36° C. | >300 sec | >300 sec |

### Examples 2a and 2b

### Manufacturing Procedure

Preparation of aerosol emulsion concentrates of example:

1) Heat approximately 90 percent of the water to about 70° C.;
2) Add Carbomer to the water with moderate agitation and continue to mix until Completely dispersed (about one hour);
3) While maintaining 70° C., add the mixture of sodium Lauryl sulfate, disodium EDTA and methylparaben;
4) Combine the petrolatum, steareth-2, steareth-20, propylparaben, Cetyl alcohol and the cyclomethicone and heat to 70° C. with low speed mixing until this phase is melted;
5) Slowly add the above oil phase to the aqueous phase with high shear agitation (Homo-mixer). After the emulsion forms, add the triethanolamine;
6) Allow the batch to cool to 50° C. with continued homomixing and then add the imidazolidinyl urea and the remaining water;
7) At 50° C. or below sprinkle in the aluminum starch octenylsuccinate as the batch cools further. Allow the batch to cool to 30° C. with low speed mixing; and
8) API was added with stirring at about 30° C. to about 40° C. depending on the API and allowed to cool.

### Example 2a

### Foamable Pharmaceutical Emulsion Compositions with 25% Petrolatum as Unctuous Emollient, Steareth-2/Steareth-211 ASOS as Multi-Active Component and Acyclovir, Azelaic Acid, Betamethasone 17 Valerate Micronized, or Caffeine as API

| | | | Betamethaso | |
|---|---|---|---|---|
| | | | valerate | |
| | | | 17 ne | |
| | Acyclovir | acid Azelaic | micronized | caffeine |
| Water | 58.55 | 48.55 | 63.43 | 58.55 |
| Carbomer 941 | 0.12 | 0.12 | 0.12 | 0.12 |
| sodium Lauryl | 0.10 | 0.10 | 0.10 | 0.10 |
| sulfate | | | | |
| Disodium EDTA | 0.05 | 0.05 | 0.05 | 0.05 |
| Methylparaben | 0.20 | 0.20 | 0.20 | 0.20 |
| Petrolatum | 25.00 | 25.00 | 25.00 | 25.00 |
| Stearath-2 | 1.73 | 1.73 | 1.73 | 1.73 |
| Steareth-20 | 0.58 | 0.58 | 0.58 | 0.58 |
| Propylparaben | 0.15 | 0.15 | 0.15 | 0.15 |
| Cetyl alcohol | 0.10 | 0.10 | 0.10 | 0.10 |
| Cyclomethicone | 5.00 | 5.00 | 5.00 | 5.00 |
| Aluminum starch | 3.00 | 3.00 | 3.00 | 3.00 |
| octenylsuccinate | | | | |
| Imidazolidinyl urea | 0.30 | 0.30 | 0.30 | 0.30 |
| Triethanolamine | 0.12 | 0.12 | 0.12 | 0.12 |
| API | 5.00 | 15.00 | 0.12 | 5.00 |
| Total | 100.00 | 100.00 | 100.00 | 100.00 |
| Propellant | 8.00 | 8.00 | 8.00 | 8.00 |
| (Propane/Butane/ | | | | |
| Isobutane) | | | | |
| FOAM QUALITY | E | G | E | G |
| ODOR | W | W | W | W |
| COLOR | V.F.O | V.F.O | V.F.O | V.F.O |
| SHAKABILITY | GOOD | MODERATE | GOOD | MOD- |
| | | | | ERATE |
| COLLAPSE | >300 | | 180 SEC. | |
| TIME AT 36° C. | | | | |
| HARDNESS | 11.71 | | 9.77 | |

### Example 2b

### Foamable Pharmaceutical Emulsion Compositions with 25% Petrolatum as Unctuous Emollient, Steareth-2/Steareth-21/ASOS as Multi-Active Component and Clindamycin Phosphate, Clotrimazole, Diclofenac Sodium, Lidocaine Base or Terbinafine HCL as API

| | Clindamicin | | Diclofenac | Lidocain | terbinafi |
|---|---|---|---|---|---|
| | Phosphate | clotrimazole | sodium | base e | HCl ne |
| Water | 62.55 | 62.55 | 62.55 | 61.55 | 62.55 |
| Carbomer 941 | 0.12 | 0.12 | 0.12 | 0.12 | 0.12 |
| sodium Lauryl sulfate | 0.10 | 0.10 | 0.10 | 0.10 | 0.10 |
| Disodium EDTA | 0.05 | 0.05 | 0.05 | 0.05 | 0.05 |
| Methylparaben | 0.20 | 0.20 | 0.20 | 0.20 | 0.20 |
| Petrolatum | 25.00 | 25.00 | 25.00 | 25.00 | 25.00 |
| Stearath-2 | 1.73 | 1.73 | 1.73 | 1.73 | 1.73 |
| Steareth-20 | 0.58 | 0.58 | 0.58 | 0.58 | 0.58 |
| Propylparaben | 0.15 | 0.15 | 0.15 | 0.15 | 0.15 |
| Cetyl alcohol | 0.10 | 0.10 | 0.10 | 0.10 | 0.10 |
| Cyclomethicone | 5.00 | 5.00 | 5.00 | 5.00 | 5.00 |
| Aluminum | 3.00 | 3.00 | 3.00 | 3.00 | 3.00 |
| starch octenylsuccinate | | | | | |
| Imidazolidinyl urea | 0.30 | 0.30 | 0.30 | 0.30 | 0.30 |
| Triethanolamine | 0.12 | 0.12 | 0.12 | 0.12 | 0.12 |
| API | 1.00 | 1.00 | 1.00 | 2.00 | 1.00 |
| Total | 100.00 | 100.00 | 100.00 | 100.00 | 100.00 |
| Propellant | 8.00 | 9.00 | 10.00 | 10.00 | 10.00 |
| (Propane/Butane/Isobutane) | | | | | |
| FOAM | G | E | G | FG | E |
| QUALITY | | | | | |
| ODOR | W | W | W | W | W |
| COLOR | V.F.O | V.F.O | V.F.O | V.F.O | V.F.O |
| SHAKABILITY | GOOD | GOOD | GOOD | GOOD | GOOD |
| COLLAPSE | | >300 | | | |
| TIME AT 36° C. | | | | | |
| HARDNESS | | 15.38 | | | |

### Example 3a

### Foamable Pharmaceutical Emulsion Compositions with 12.5% Petrolatum as Unctuous Emollient, Steareth-2/Steareth-21/ASOS as Multi-Active Component and Acyclovir, Azelaic Acid, Betamethasone 17 Valerate Micronized, or Caffeine as API

| | | | 17 Beta- | |
|---|---|---|---|---|
| | | | methasone | |
| | | | micronized | |
| | Acyclovir | acid Azelaic | valerate | caffeine |
| Water | 58.55 | 61.05 | 75.93 | 71.05 |
| Carbomer 941 | 0.12 | 0.12 | 0.12 | 0.12 |
| sodium Lauryl | 0.10 | 0.1 | 0.1 | 0.1 |
| sulfate | | | | |
| Disodium EDTA | 0.05 | 0.05 | 0.05 | 0.05 |
| Methylparaben | 0.20 | 0.2 | 0.2 | 0.2 |
| Petrolatum | 25.00 | 12.5 | 12.5 | 12.5 |
| Stearath-2 | 1.73 | 1.73 | 1.73 | 1.73 |
| Steareth-20 | 0.58 | 0.58 | 0.58 | 0.58 |
| Propylparaben | 0.15 | 0.15 | 0.15 | 0.15 |
| Cetyl alcohol | 0.10 | 0.1 | 0.1 | 0.1 |
| Cyclomethicone | 5.00 | 5 | 5 | 5 |
| Aluminum starch | 3.00 | 3 | 3 | 3 |
| octenylsuccinate | | | | |
| Imidazolidinyl urea | 0.30 | 0.3 | 0.3 | 0.3 |
| Triethanolamine | 0.12 | 0.12 | 0.12 | 0.12 |
| API | 5.00 | 15 | 0.12 | 5 |
| Total | 100.00 | 100 | 100 | 100 |
| Propellant | 8.00 | 8 | 8 | 8 |
| (Propane/Butane/ | | | | |
| Isobutane) | | | | |
| FOAM QUALITY | E | G | E | FG |
| ODOR | W | W | W | W |
| COLOR | V.F.O | V.F.O | V.F.O | V.F.O |
| SHAKABILITY | GOOD | MODERATE | GOOD | MOD- |
| | | | | ERATE |
| COLLAPSE TIME | 110 | | >300 SEC. | |
| AT 36° C. | | | | |
| HARDNESS | 5.36 | | 10.47 | |

### Example 3b

### Foamable Pharmaceutical Emulsion Compositions with 12.5% Petrolatum as Unctuous Emollient, Steareth-2/Steareth-21/ASOS as Multi-Active Component and Clindamycin Phosphate, Clotrimazole, Diclofenac Sodium, Lidocaine Base or Terbinafine HCL as API

| | Clindamicin | | Diclofenac | Lidocain | terbinafi |
|---|---|---|---|---|---|
| | Phosphate | clotrimazole | sodium | base e | HCl ne |
| Water | 75.05 | 75.05 | 75.05 | 74.05 | 75.05 |
| Carbomer 941 | 0.12 | 0.12 | 0.12 | 0.12 | 0.12 |
| sodium Lauryl | 0.1 | 0.1 | 0.1 | 0.1 | 0.1 |
| sulfate | | | | | |
| Disodium EDTA | 0.05 | 0.05 | 0.05 | 0.05 | 0.05 |
| Methylparaben | 0.2 | 0.2 | 0.2 | 0.2 | 0.2 |
| Petrolatum | 12.5 | 12.5 | 12.5 | 12.5 | 12.5 |
| Stearath-2 | 1.73 | 1.73 | 1.73 | 1.73 | 1.73 |
| Steareth-20 | 0.58 | 0.58 | 0.58 | 0.58 | 0.58 |
| Propylparaben | 0.15 | 0.15 | 0.15 | 0.15 | 0.15 |
| Cetyl alcohol | 0.1 | 0.1 | 0.1 | 0.1 | 0.1 |
| Cyclomethicone | 5 | 5 | 5 | 5 | 5 |
| Aluminum starch | 3 | 3 | 3 | 3 | 3 |
| octenylsuccinate | | | | | |
| Imidazolidinyl urea | 0.3 | 0.3 | 0.3 | 0.3 | 0.3 |
| Triethanolamine | 0.12 | 0.12 | 0.12 | 0.12 | 0.12 |
| API | 1 | 1 | 1 | 2 | 1 |
| Total | 100 | 100 | 100 | 100 | 100 |
| Propellant | 8 | 9 | 10 | 10 | 10 |
| (Propane/Butane/Isobutane) | | | | | |
| FOAM QUALITY | FG | E | FG | FG | G |
| ODOR | W | W | W | W | W |
| COLOR | V.F.O | V.F.O | V.F.O | V.F.O | V.F.O |
| SHAKABILITY | GOOD | GOOD | GOOD | GOOD | GOOD |
| HARDNESS | | 9.69 | | | |

### Example 4

### Foamable Pharmaceutical Emulsion Compositions with 12.5% or 25% Petrolatum as Unctuous Emollient, Steareth-2/Steareth-21 as Multi-Active Component With and without Clindamycin as API

| | Ingredient name | | | | | |
|---|---|---|---|---|---|---|
| Phase | (INCI, CTFA) | | % w/w | % w/w | % w/w | % w/w |
| Water phase (A) | Water | | 70 | 69 | 82.5 | 81.5 |
| Oil phase (B) | Petrolatum | | 25 | 25 | 12.5 | 12.5 |
| | Steareth-2 | | 2 | 2 | 2 | 2 |
| | Steareth-21 | | 3 | 3 | 3 | 3 |
| API | Clindamycin Phosphate | | | 1 | | 1 |
| TOTAL | | | 100 | 100 | 100 | 100 |
| Propellant | | | 8 | 8 | 8 | 8 |
| (Propane/Butane | | | | | | |
| Isobutane) | | | | | | |
| ZT | APPEARANCE | QUALITY | G | FG | G | FG |
| | | COLOR | W | W | W | W |
| | | ODOR | V.F.O | V.F.O | V.F.O | V.F.O |

| | | | |
|---|---|---|---|
| COLLAPSE TIME | >300 | 180 | |
| HARDNESS | 7.84 | 6.2 | 4.74 |

The placebo formula with 25% petrolatum underwent in a separate test two FTC cycles without change in foam appearance demonstrating the stability of the carrier.

### Example 5

### Foamable Pharmaceutical Emulsion Compositions with 12.5% or 25% Petrolatum as Unctuous Emollient, Steareth-2/Steareth-21/CBC and ASOS as Multi-Active Component with and without Clindamycin as API

| | Ingredient name | | | | | |
|---|---|---|---|---|---|---|
| Phase | (INCI, CTFA) | | % w/w | % w/w | % w/w | % w/w |
| Water phase (A) | Water | | 67 | 66 | 79.5 | 78.5 |
| Oil phase (B) | Petrolatum | | 25 | 25 | 12.5 | 12.5 |
| | Steareth-2 | | 2 | 2 | 2 | 2 |
| | Steareth-21 | | 3 | 3 | 3 | 3 |
| | Aluminum starch | | 3 | 3 | 3 | 3 |
| | octenylsuccinate | | | | | |
| API | Clindamycin Phosphate | | | 1 | | 1 |
| TOTAL | | | 100 | 100 | 100 | 100 |
| Propellant | | | 8 | 8 | 8 | 8 |
| (Propane/Butane/ | | | | | | |
| Isobutane) | | | | | | |
| ZT | COLLAPSE TIME | | >300 | >300 | >300 | >300 |
| | HARDNESS | | 14.78 | 8.28 | 17.16 | 14.96 |
| | APPEARANCE | QUALITY | E | E | E | E |
| | | COLOR | W | W | W | W |
| | | ODOR | V.F.O | V.F.O | V.F.O | V.F.O |

The placebo formulas with 25% petrolatum and 12/5% petrolatum underwent in a separate test two FTC cycles without change in foam appearance demonstrating the stability of the carrier foam and the API foam. In a further test a placebo successfully underwent three cycles again with no significant appearance change.

### Example 6

Preparation of aerosol emulsion concentrates of Example 6:

1) Heat approximately 90 percent of the water to about 70° C.;
2) Combine the petrolatum, steareth-2, steareth-21 and heat to 70° C. with low speed mixing until this phase is melted;
3) Slowly add the water phase to the oil with high shear agitation;
4) Allow the batch to cool to 50° C. with continued homogenizing and then add the remaining water;
5) At 50° C. or below sprinkle in the aluminum starch octenylsuccinate as the batch cools further. Allow the batch to cool to 30° C. with low speed mixing; and
6) Split the emulsion to two portions of 200 g.

| | | | Actual | | | | Actual |
|---|---|---|---|---|---|---|---|
| PLACEBO | w/w | Amount | weight | API | w/w | Amount | weight |
| Stock emulsion | 99.00 | 198.00 | | Stock | 99.00 | 198.00 | |
| | | | | emulsion | | | |
| Water | 1.00 | 2.00 | | Clindamycin | 1.00 | 2.00 | |
| | | | | Phosphate | | | |
| Propellant | 8.00 | 8.00 | | | 8.00 | 8.00 | |
| (Propane/ | | | | | | | |
| Butane/Isobutane) | | | | | | | |

Add Clindamycin Phosphate to Stock solution at RT and mix until completely dissolves.

### Example 7

Preparation of aerosol emulsion concentrates of Example 7:

1) Heat approximately 90 percent of the water to about 70° C. Add Avicel with high speed mixing until uniformity;
2) Combine the petrolatum, steareth-2, steareth-21 and heat to 70° C. with low speed mixing until this phase is melted;
3) Slowly add the oil phase to the water with high shear agitation;
4) Allow the batch to cool to 50° C. with continued homogenizing and then add the remaining water;
5) Allow the batch to cool to 30° C. with low speed mixing; and
6) Continue as describes in the table below:

| | | | Actual | | | | Actual |
|---|---|---|---|---|---|---|---|
| PLACEBO | w/w | Amount | weight | API | w/w | Amount | weight |
| Stock emulsion | 99.00 | 198.00 | | Stock | 99.00 | 198.00 | |
| | | | | emulsion | | | |
| Water | 1.00 | 2.00 | | Clindamycin | 1.00 | 2.00 | |
| | | | | Phosphate | | | |
| Propellant | 8.00 | 8.00 | | | 8.00 | 8.00 | |
| (Propane/Butane/ | | | | | | | |
| Isobutane) | | | | | | | |

Add Clindamycin Phosphate to Stock solution at RT and mix until completely dissolves.

### Example 8

### Foamable Pharmaceutical Emulsion Compositions with 25% Petrolatum as Unctuous Emollient, Steareth-2/Steareth-211 CBC and MCC as Multi-Active Component With and without Clindamycin as API

| | Ingredient name | | | |
|---|---|---|---|---|
| Phase | (INCI, CTFA) | | % w/w | % w/w |
| Water phase (A) | Water | | 67 | 66 |
| | Carboxymethyl cellulose | | 3 | 3 |
| | sodium | | | |
| | and microcrystalline | | | |
| | cellulose | | | |
| Oil phase (B) | Petrolatum | | 25 | 25 |
| | Steareth-2 | | 2 | 2 |
| | Steareth-21 | | 3 | 3 |
| API | Clindamycin Phosphate | | | 1 |
| TOTAL | | | 100 | 100 |
| Propellant | | | 8 | 8 |
| (Propane/Butane/ | | | | |
| Isobutane) | | | | |
| ZT | APPEARANCE | QUALITY | G | G |
| | | COLOR | W | W |
| | | ODOR | V.F.O | V.F.O |

| | | |
|---|---|---|
| COLLAPSE TIME | >300 | >301 |
| HARDNESS | 13.58 | 14.85 |

### Example 9

### Foamable Pharmaceutical Emulsion Compositions with 25% Petrolatum as Unctuous Emollient, Arlacel 2121/Sucrose Stearate and ASOS as Multi-Active Component with and without Clindamycin as API

| | Ingredient name | | | |
|---|---|---|---|---|
| Phase | (INCI, CTFA) | | % w/w | % w/w |
| Water | Water | | 68 | 67 |
| phase (A) | | | | |
| Oil phase | Petrolatum | | 25 | 2 |
| (B) | Arlacel 2121 | | 2 | 25 |
| | Sucrose stearate | | 2 | 2 |
| | Aluminum starch | | 3 | 3 |
| | octenylsuccinate | | | |
| API | | | | 1 |
| | Total | | 100 | 100 |
| ZT | APPEARANCE | QUALITY | E | E |
| | | COLOR | W | W |
| | | ODOR | N.O | N.O |

### Example 10

### Transdermal Water Loss

| INCI/CTFA Name | % w/w | % w/w | % w/w | % w/w | % w/w |
|---|---|---|---|---|---|
| Octyl Dodecanol | | | 12.00 | 12.00 | |
| Cetearyl Octanoate | | | | | |
| Vaseline | | 22.00 | 61 | | |
| Mineral oil heavy | 22.00 | | | | |
| Isopropyl myristate | 11.00 | 11.00 | 11.00 | 21.00 | |
| Isopropyl Palmitate | | | | | |
| Benzyl alcohol | 1.50 | 1.50 | 1.50 | 1.50 | 1.50 |
| Glyceryl | 0.50 | 0.50 | 0.50 | 0.50 | 0.50 |
| monostearate | | | | | |
| Ceteareth-20 | 3.30 | 3.30 | 3.30 | 3.30 | 3.30 |
| Stearyl alcohol | 1.10 | 1.10 | 1.10 | 1.10 | 1.10 |
| Permethrin | 5.05 | 5.05 | 5.05 | 5.05 | 5.05 |
| Water, purified | 51.70 | 51.70 | 61.70 | 51.70 | 84.70 |
| Carboxymethyl | 0.55 | 0.55 | 0.55 | 0.55 | 0.55 |
| cellulose | | | | | |
| Glycerin | 3.30 | 3.30 | 3.30 | 3.30 | 3.30 |
| Total product: | 100.00 | 100.00 | 100.00 | 100.00 | 100.00 |
| T-0 | 4.68 | 4.53 | 4.44 | 4.00 | 4.38 |
| T-60 | 2.67 | 2.84 | 2.49 | 2.24 | 2.44 |
| T-120 | 3.32 | 3.34 | 3.07 | 3.10 | 3.10 |

The results show non-occlusiveness of the formulations.

### Example 11

### Exemplary Foamable Carrier with 20% Petrolatum as Unctuous Emollient and Cetearyl Glycoside or Sorbitan Stearate,/Arlacel 2121 and Methocel K100M/Xanthan Gum or CMC or ASOS as Multi-Active Component

| | % w/w | % w/w | % w/w | % w/w | % w/w | % w/w |
|---|---|---|---|---|---|---|
| Petrolatum | 20.00 | 20.00 | 20.00 | 20.00 | 20.00 | 20.00 |
| Cetearyl | 3.00 | — | 3.00 | — | 3.00 | — |
| glycoside | | | | | | |
| Sorbitan | | 2.00 | | 2.00 | | 2.00 |
| stearate | | | | | | |
| Arlacel 2121 | | 3.00 | | 3.00 | | 3.00 |
| Methocel | 0.30 | 0.30 | — | — | — | 0.30 |
| K100M | | | | | | |
| Xanthan gum | 0.30 | 0.30 | — | — | — | — |
| CMC | — | — | 1.00 | 1.00 | — | 3.00 |
| ASOS | | | | | 3.00 | |
| Water, purified | 71.40 | 69.40 | 71.00 | 69.00 | 69.00 | 66.70 |
| Propellant | 5.00 | 5.00 | 5.00 | 5.00 | 5.00 | 5.00 |
| Control: | 100.00 | 100.00 | 100.00 | 100.00 | 100.00 | 100.00 |

Note:
Each of these exemplary carriers in example 10 is believed capable of producing a good quality foam which can carry an API, which has a degree of solubility, a limited degree of solubility or is particulate, as described herein.

### Example 12

Exemplary concentrations of active agents in foamable compositions are set out in Table 2. Each active agent is added into, for example, any of the carriers listed in any of Examples 1 to 8 above in a therapeutically effective concentration and amount. The methodology of addition is well known to those of the art. The composition is adjusted in each case so that it is made up to 100% w/w as appropriate by purified water.

TABLE 2
Exemplary Concentrations of Examples of Active Agents

| Class | Concentration | Exemplary Use |
|---|---|---|
| Hydrocortisone | 1% | Steroid responsive inflammation and |
| acetate | | |
| Betamethasone | 0.12% | psoriasis or atopic dermatitis |
| valerate | | |
| Clobetasol | 0.05% | |
| proprionate | | |
| Acyclovir | 5% | Viral infection, herpes |
| Ciclopirox | 1% | Fungal infection, seborrhea, dandruff, |
| Clindamycin | 1-2% | Bacterial infection, acne, rosacea, |
| Azelaic acid | 15% | Acne, rosacea, |
| | | pigmentation disorder and various |
| | | dermatoses |
| Metronidazol | 0.25%-2% | Rosacea, bacterial infections and |
| | | parasite infestations |
| Diclofenac | 1% | Osteoarthritus, joint pain |
| Tacrolimus | 0.2% | Atopic dermatitis, eczema and |
| | | inflammation |
| Caffeine | 5% | anti-cellulite |
| Clotrimazole | 1% | Fungal infection |
| Lidocaine base | 2% | Local anaesthetic |
| Terbinafine HCL | 1% | Fungal infection |
| Gentamycin | 0.1% | Bacterial skin infections, burns or |
| | | ulcers |
| Dexpanthenol | 5% | Wounds, ulcers, minor skin infections |
| Urea | 5-10% | Emollient and keratolytic |
| | | Atopic dermatitis, eczema, ichthyosis |
| | | and hyperkeratotic skin disorders |
| Ammonium | 12%-17.5% | Dry scaly conditions of the skin |
| lactate | | including ichthyosis |
| Povidone-iodine | 10% | Antimicrobial - antiseptic |

Note, all the above active agents have a degree of solubility in water or petrolatum or the composition other than clobestol proprionate, which is practically insoluble; tacrolimus, which is insoluble in water; and betamethasone valerate which although has very limited solubility is nevertheless, surprisingly soluble at least to a degree in the compositions of the present invention, in the water phase.

The above examples represent different drug classes and it is to be understood that other drugs belonging to each of the classes represented above or described elsewhere in the specification may be included and used in the compositions of the present invention in a safe and effective amount.

### Part B

### Example 13

### Complex Emulgators with 49% Petrolatum, where the Difference in HLB is in Excess of 10

| | HLB/ | | | |
|---|---|---|---|---|
| | RHLB | EPP001 | EPP002 | EPP005 |
| chemical name | | | | |
| white petrolatum | 7.0 | 49.00 | 49.00 | 49.00 |
| (sofmetic) | | | | |
| Sreareth 2 | 4.9 | 7.00 | | |
| Sreareth 21 | 15.5 | 2.00 | | |
| Sorbitan oleate | 4.3 | | 6.00 | |
| Polysorbate 60 | 14.9 | | 2.00 | |
| Sorbitan | 6.7 | | | |
| monopalmitate | | | | |
| Methocel K100M | | | | 0.25 |
| Xanthabn gum | | | | 0.25 |
| Water | | 42.00 | 43.00 | 50.50 |
| Total | | 100.00 | 100.00 | 100.00 |
| Propellant (AP 70) | | 8.00 | 8.00 | 8.00 |
| Results: | | | | |
| viscosity | | 170963.30 | 156126.68 | 82702.35/ |
| (1 RPM/10 RPM) | | | | 17852.19 |
| foam quality | | G | G | G |
| Color | | White | White | White |
| Odor | | No odor | No odor | No odor |
| Shakability | | Moderate | Good | Poor |
| Density | | 0.053 | 0.123 | |
| collapse time | | >300/G | >300/FG | >300/G |
| Hardness | | 36.82 | 20.20 | 25.43 |
| Bubble Mean Size | | 181 | 171 | 220 |
| (μm) | | | | |
| Bubbles Over 500 | | N/R | N/R | 7.1 |
| μm (%) | | | | |
| FTC: | | | | |
| foam quality | | G | G | FG |
| Color | | White | White | White |
| Odor | | No odor | No odor | No odor |
| Shakability | | Moderate | Good | Good |

### Comments:

By selecting a complex emulgator as multi active agent where the difference in HLB is in excess of 10 it was possible to achieve a soft stable breakable foam able to withstand FTC having a relatively low hardness, even though the prefoam formulation has a high viscosity. On the other hand using, Avicel (2%); or Carbopol (0.5%); or Permulen (0.25%) alone with 49% petrolatum only produced poor foam with phase separation; whilst carboxymethyl cellulose, a polymeric agent, alone with 49% petrolatum at best (0.5%) produced fairly good foam. Using a combination of polymeric agents, Methocel K100M and Xanthabn gum, was more successful but the product had poor shakability and quality deteriorated after FTC. Measuring bubble size in formulations with high petrolatum is not straightforward and should be repeated.

### Example 14

### Focus Group on the Look and Feel of Complex Emulgators with 45% Petrolatum where the Difference in HLB is in Excess of 10 with and without ASOS Compared to Polymer Alone or to Petrolatum Alone

### a) Formulations

| | HLB/ | | | |
|---|---|---|---|---|
| | RHLB | EPP010 | EPP011 | EPP012 |
| chemical name | | | | |
| white petrolatum | 7.0 | 45.00 | 45.00 | 45.00 |
| (sofmetic) | | | | |
| Sorbitan oleate | 4.3 | 6.00 | | 6.00 |
| Polysorbate 60 | 14.9 | 2.00 | | 2.00 |
| Sorbitan monopalmitate | 6.7 | | | |
| Methocel K100M | | | 0.25 | |
| Xanthabn gum | | | 0.25 | |
| ASOS | | | | 3.00 |
| Water | | 47.00 | 54.50 | 44.00 |
| Total | | 100.00 | 100.00 | 100.00 |
| Propellant (AP 70) | | 8.00 | 8.00 | 8.00 |
| | | focus group | | |
| Results: | | | | |
| foam quality | | G | G | G |
| Color | | White | White | White |
| Odor | | v.f. odor | v.f. odor | v.f. odor |
| Shakability | | Good | Good | Good |
| Density | | 0.17 | 0.058 | 0.17 |
| collapse time | | >300/FG | >300/G | >300/FG |

### b) (i) Focus Group Results

| | EPP010 | | | EPP011 | | | EPP010 | | |
|---|---|---|---|---|---|---|---|---|---|
| | Total = 130 for all 10 | | | Total = 169 for all 10 | | | Total = 155 for all 10 | | |
| | categories | | | categories | | | categories | | |
| | Total | | | Total | | | Total | | |
| | points | Avg/5 | STDV | points | Avg/5 | STDV | points | Avg/5 | STDV |
| spreadability | 12 | 2.4 | 1.14 | 17 | 3.4 | 1.52 | 14 | 2.8 | 1.10 |
| skin feeling | 16 | 3.2 | 1.48 | 17 | 3.4 | 0.55 | 15 | 3 | 1.22 |
| stability on | 20 | 4 | 1.00 | 20 | 4 | 1.00 | 19 | 3.8 | 1.30 |
| skin | | | | | | | | | |
| Absorption | 7 | 1.4 | 0.55 | 16 | 3.2 | 1.30 | 12 | 2.4 | 0.89 |
| (1 min). | | | | | | | | | |
| uniformity | 15 | 3 | 1.41 | 17 | 3.4 | 1.52 | 18 | 3.6 | 0.89 |
| Greasy | 9 | 1.8 | 1.10 | 17 | 3.4 | 1.14 | 14 | 2.8 | 0.45 |
| feeling (after | | | | | | | | | |
| 1-2 min.) | | | | | | | | | |
| Shiny look | 10 | 2 | 1.00 | 17 | 3.4 | 1.52 | 15 | 3 | 0.00 |
| (after 1-2 min.) | | | | | | | | | |
| Stickiness | 12 | 2.4 | 1.34 | 13 | 2.6 | 1.67 | 15 | 3 | 0.71 |
| (after 1-2 min.) | | | | | | | | | |
| Odor | 17 | 3.4 | 1.52 | 17 | 3.4 | 1.82 | 19 | 3.8 | 1.64 |
| total | 12 | 2.4 | 1.34 | 18 | 3.6 | 1.14 | 14 | 2.8 | 0.45 |
| satisfaction | | | | | | | | | |

As can be seen from the above the presence of the polymeric combination of methocel and xantham gum in a 50:50 ratio had the most significant effect in improving the look and feel of the composition and better than the derivatized polymer ASOS. It follows that using a multi active agent providing both varied surfactant properties and/or a polymer combination producing varied stabilizing and thickening properties is desirable to provide both good, soft, stable, flowable foam that has a satisfactory look and feel. However, polymeric agent should be used sparingly otherwise the composition will be too viscous. When the above were compared with softmetic petroleum alone formulations 10 and 11 had a substantially better points profile, which is reasonable indicator that improved skin feeling is contributed to from the presence of polymeric agent be it in the above examples, methocel/xantham or ASOS.

### b) (ii) Focus Group Results

| | EPP010 | | |
|---|---|---|---|
| | Total = | | |
| | 136 for all | | |
| | 10 | | |
| | categories | | |
| | Total | | |
| | points | Avg/5 | STDV |
| spreadability | 18 | 3.6 | 1.14 |
| skin feeling | 16 | 3.2 | 1.30 |
| stability on | 14 | 2.8 | 1.30 |
| skin | | | |
| Absorption | 11 | 2.2 | 1.10 |
| (1 min). | | | |
| uniformity | 16 | 3.2 | 1.64 |
| Greasy | 11 | 2.2 | 1.64 |
| feeling (after | | | |
| 1-2 min.) | | | |
| Shiny look | 9 | 1.8 | 1.30 |
| (after 1-2 min.) | | | |
| Stickiness | 11 | 2.2 | 1.64 |
| (after 1-2 min.) | | | |
| odor | 18 | 3.6 | 1.67 |
| total | 12 | 2.4 | 1.67 |
| satisfaction | | | |

### Example 15

### Single Agent With and Without Ciclopiroxolamine and 49% or 45% Petrolatum

| | EPP013 | EPP014 | EPP015 | EPP009 | EPP016 |
|---|---|---|---|---|---|
| chemical name | | | | | |
| white petrolatum | 49.00 | 49.00 | 45.00 | 45.00 | 45.00 |
| (sofmetic) | | | | | |
| Sorbitan monopalmitate | 3.00 | 3.00 | 2.00 | 5.00 | 10.00 |
| Water | 47.00 | 47.00 | 53.00 | 50.00 | 45.00 |
| Ciclopiroxolamine | 1.00 | 1.00 | | | |
| NaOH solution | | to ph = 7 | | | |
| Total | 100.00 | 100.00 | 100.00 | 100.00 | 100.00 |
| Propellant (AP 70) | 8.00 | 8.00 | 8.00 | 8.00 | 8.00 |
| Results: | | | | | |
| viscosity (1 RPM/ | 12333.37 | | 201716.95 | 6750.56 | 517323.56/ |
| 10 RPM) | | | | | 0.5 rpm |
| foam quality | G | G | G | G | G |
| Color | White | White | White | White | White |
| Odor | v.f. odor | v.f. odor | no odor | v.f. odor | no odor |
| Shakability | Good | Good | good | Good | moderate |
| collapse time | >300/G | >300/G | >300/G | | >300/G |
| Hardness | 34.10 | | | | |
| Bubble Mean Size (μm) | 173 | | | 255.118 | |
| Bubbles Over 500 μm | 0 | | | 6 | |
| (%) | | | | | |
| FTC: | | | | | |
| foam quality | G | | | | |
| Color | White | | | | |
| Odor | No odor | | | | |
| Shakability | Good | | | | |

Comment: Remarkably it was possible to achieve good quality soft breakable foam stable to FTC using a single solid agent with and without an active pharmaceutical agent, ciclopiroxolamine an antimicrobial that is slight soluble in water. The effect of modulating the pH to agent and of progressively increasing the amount of agent is demonstrated. The selection of single agent reflects the absence of a polymeric element and molecular structure so it is more likely to mix with petrolatum and water and its HLB being very close to that required by petrolatum.

### Example 16

### Single Agent with Coal Tar and 49% Petrolatum

| | HLB/ | | |
|---|---|---|---|
| | RHLB | EPP017 | EPP017A |
| chemical name | | | |
| white petrolatum (sofmetic) | 7.0 | 45.00 | 45.00 |
| Sorbitan monopalmitate | 6.7 | 5.00 | 5.00 |
| Water | | 40.00 | 48.00 |
| LCD | | 10.00 | 2* |
| Total | | 100.00 | 98.00 |
| Propellant (AP 70) | | 8.00 | 8.00 |
| Results: | | | |
| viscosity(1 RPM/10 RPM) | | 13245.17 | 13677.08 |
| foam quality | | G | G |
| Color | | Yellow | Yellow |
| Odor | | character.odor | character.odor |
| shakability | | moderate | moderate |
| Density | | 0.115 | 0.305 |
| collapse time | | >300/G | >300/G |
| Hardness | | 28.14 | 39.91 |
| Bubble Mean Size (μm) | | 86 | 116 |
| Bubbles Over 500 μm (%) | | 0.0 | 0.0 |

*ALCOHOL EVAPORATED

Comment: Coal tar is miscible in Petrolatum. These formulations are based on Formulation 9, Example 14. The addition of a coal tar alcoholic extract (LCD ˜20% coal tar and 80% ethanol) effected the color of the pre foam formulation. Removal of the alcohol resulted in the color being a little more pronounced and glossier. However upon conversion to foam the preparations surprisingly became off white. The elimination of alcohol substantially affected the foam density. Using one surfactant having HLB similar to RHLB of petrolatum performs good, stable foam and PFF having small bubble size

### Example 17

### Single Agent with CoalTar Extract combined with another Active Agent and about 45% Petrolatum

a)

| | EPP017 | EPP017B | EPP017C | EPP017D |
|---|---|---|---|---|
| chemical name | | | | |
| white petrolatum | 45.00 | 44.96 | 44.98 | 44.98 |
| (sofmetic) | | | | |
| Sorbitan | 5.00 | 5.00 | 5.00 | 5.00 |
| monopalmitate | | | | |
| Water | 40.00 | 39.96 | 39.98 | 39.98 |
| LCD | 10.00 | 9.99 | 10.00 | 10.00 |
| Triamcinolone | | 0.10 | | |
| Clobetasol | | | 0.05 | |
| propionate | | | | |
| Calcipotriol | | | | 0.05 |
| Total | 100.00 | 100.00 | 100.00 | 100.00 |
| Propellant | 8% | 8% | 8% | 8% |
| Results: | | | | |
| Viscosity | | | | 12077.42 |
| foam quality | | | | GOOD |
| Color | | | | Yellow |
| Odor | | | | character.odor |
| Shakability | | | | GOOD |
| Density | | | | 0.090 |
| Bubble Mean Size | | | | 81 |
| (μm) | | | | |
| Bubbles Over 500 μm | | | | 0 |
| (%) | | | | |
| Collapse Time (sec) | | | | >300 |

b)

| | EPP017E | EPP017F | EPP017G | EPP017H | EPP017I |
|---|---|---|---|---|---|
| chemical name | | | | | |
| white petrolatum | 44.10 | 44.55 | 44.55 | 44.55 | 44.55 |
| (sofmetic) | | | | | |
| Sorbitan | 4.90 | 4.95 | 4.95 | 4.95 | 4.95 |
| monopalmitate | | | | | |
| Water | 39.20 | 39.60 | 39.60 | 39.60 | 39.60 |
| LCD | 9.80 | 9.90 | 9.90 | 9.90 | 9.90 |
| Salycilic acid | 2.00 | | | | |
| Diclofenac sodioum | | 1.00 | | | |
| Pimicrolimous | | | 1.00 | | |
| Ketoconazole | | | | 1.00 | |
| Ciclopiroxolamine | | | | | 1.00 |
| Total | 100.00 | 100.00 | 99.00 | 100.00 | 100.00 |

Comment: It is possible to make a wide variety of foams of good quality comprising two or more active agents one of which is coal tar extract and the other also being a dermatological agent. It is further possible to do so perhaps due to the excellent miscible properties of the agent with petrolatum, its very close proximity to the required HLB, and its medium fatty acid chain, also perhaps coupled to the good miscibility of coal tar in petrolatum.

### Part C

### Example C1

### Petrolatum and Mineral Oil in a ratio of about 7:3 with a Complex of Two Surfactants, a Wax and an Emollient Like Foam Adjuvant

| Ingredients | TLF013 | TLF013 |
|---|---|---|
| White Petrolatum (sofmetic) | 42.00 | 42.00 |
| Mineral oil, light | 18.00 | 18.00 |
| Cetearyl alcohol | 2.00 | 2.00 |
| Ceteth-20 (Lipocol C-20) | 2.16 | 2.16 |
| Span 80 | 3.84 | 3.84 |
| Behenyl alcohol | 1.00 | 1.00 |
| Aluminum starch octenyl succinate | 3.00 | 3.00 |
| Citric acid | 0.18 | 0.18 |
| Sodium citrate | 0.14 | 0.14 |
| Water, purified | 27.48 | 27.48 |
| Preservative | 0.20 | 0.20 |
| Total | 100.00 | 100.00 |
| Propellant type | 10% 1681 | 10% Dymel |
| | | 134A |
| Quality | G-E | E |
| Shakability | Good | good |
| Stability FTC | Stable | Stable |
| Centrifugation | 1K-stable | N/M |
| Flammability | Flammable | non flammable |

N/M = Not Measured

Comment: The formulation provides a stable unctuous composition, which when Dymel is the propellant is non flammable. Behenyl alcohol is saturated C22 fatty alcohol, which apart from having antiviral activity and acting as a co-surfactant or foam adjuvant is said to usable as a thickening agent and can, help make skin smoother and prevent moisture loss. Cetearyl Alcohol is a waxy mixture of fatty alcohols, being primarily cetyl and stearyl alcohols. It is used as an emulsion stabilizer, foam booster, and viscosity-increasing agent and it imparts an emollient feel to the skin.

### Example C2

### Petrolatum and Mineral Oil in a Ratio of About 7:3; or of About 7:5 Compared to the Same Formulation Without Mineral Oil with a Complex of Two Surfactants, a Wax and an Emollient Like Foam Adjuvant with and without ASOS

| | TLF013 | TLF023 | TLF024 |
|---|---|---|---|
| White Petrolatum (sofmetic) | 42.00 | 35.00 | 60.00 |
| light Mineral oil | 18.00 | 25.00 | |
| Cetearyl alcohol | 2.00 | 2.00 | 2.00 |
| Ceteth-20 (Lipocol C-20) | 2.16 | 2.16 | 2.16 |
| (Sorbitan oleate) Span 80 | 3.84 | 3.84 | 3.84 |
| Behenyl alcohol | 1.00 | 1.00 | 1.00 |
| Aluminum starch octenyl | 3.00 | | |
| succinate | | | |
| Citric acid | 0.18 | | |
| Sodium citrate | 0.14 | | |
| Water, purified | 27.48 | 31.00 | 31.00 |
| Preservative | 0.20 | | |
| Total | 100.00 | 100.00 | 100.00 |
| Propellant type | 10% | 10% | 10% |
| | 1681 | 1681 | 1681 |
| Quality | G-E | G-E | G-E |
| Color | White | White | White |
| Odor | No odor | No odor | No odor |
| Density | 0.065 | | |
| ph PFF diluted (1:5) | 4.04 | | |
| ph foam diluted (1:5) | 3.98 | | |
| Shakability | good | Good | Good |
| Collapse time (sec.) | >300 | | |
| Hardness (g) | 35.59 | | |
| Centrifugation 1K | | Stable | Stable |
| Viscosity (cp) | 19167 | | |
| Centrifugation 3K | | Stable | Stable |
| Centrifugation 10K | | Stable | Stable |

Comments; all three formulations shows that up to 60% oily phase in different variations, containing same surfactants in same concentration as well as same propellant, can produce good quality stable foam.

### Example C3

### 31% Petrolatum with Various Surfactants (but without Mineral Oil, a Foam Adjuvant and ASOS)

| | TLF026 | TLF028 | TLF029 | TLF038 |
|---|---|---|---|---|
| White Petrolatum (sofmetic) | 31.00 | 31.00 | 31.00 | |
| White Petrolatum (Pioner | | | | 31.00 |
| 5464) | | | | |
| Ceteth-20 (Lipocol C-20) | | | 1.00 | |
| Polysorbate 80 | | 2.00 | | 2.00 |
| Steareth-2 | 6.00 | | | |
| Steareth-21 | 2.00 | | | |
| (Sorbitan oleate) Span 80 | | 5.00 | 3.00 | 5.00 |
| Water, purified | 61.00 | 62.00 | 65.00 | 62.00 |
| Total | 100.00 | 100.00 | 100.00 | 100.00 |
| Propellant type | 10% | 10% | 10% 1681 | 10% 1681 |
| | 1681 | 1681 | | |
| Quality | G-E | G-E | G-E | G-E |
| Color | White | White | White | White |
| Odor | No odor | No odor | No odor | No odor |
| Shakability | Good | Good | Good | Good |
| Centrifugation 1K | Stable | Stable | Stable | |
| Centrifugation 3K | Stable | Stable | Stable | |
| Centrifugation 10K | Stable | Non | Non | |
| | | Stable | Stable | |
| | | | | 10% AP- |
| | | | | 70 |
| | | | | G-E |
| | | | | White |
| | | | | No odor |
| | | | | Good |

Comments; Four different complex emulgators were investigated. Three (shown above) supported good stable foam. The combination of PEG 40 stearate (2.6%) and polysorbote 80 (0.9%) (not shown) resulted in precipitation. Likewise, sucrose stearic acid esters D-1807 (3%) also resulted in precipitation. Of the three successful combinations steareth 2 and steareth 21 proved the most powerful emulgator emulsifier with the composition showing emulsion stability in the face of 10K centrifugation for 10 mins. Changing the petrolatum source and or changing the propellant did not result in a noticeable change in foam quality.

### Example C4

### 49% Petrolatum with Various Surfactants (but without Mineral Oil, a Foam Adjuvant and ASOS)

| | TLF030 | TLF031 | TLF032 |
|---|---|---|---|
| White Petrolatum (sofmetic) | 49.00 | 49.00 | 49.00 |
| Ceteth-20 (Lipocol C-20) | 2.00 | | |
| Polysorbate 80 | | | 2.00 |
| Steareth-2 | | 6.00 | |
| Steareth-21 | | 2.00 | |
| (Sorbitan oleate) Span 80 | 6.00 | | 5.00 |
| Water, purified | 43.00 | 43.00 | 44.00 |
| Total | 100.00 | 100.00 | 100.00 |
| Propellant type | 10% 1681 | 10% 1681 | 10% 1681 |
| Quality | G-E | G-E | G-E |
| Color | White | White | White |
| Odor | No odor | No odor | No odor |
| Shakability | Good | Good | Good |
| Centrifugation 1K | Stable | Stable | Stable |
| Centrifugation 3K | Stable | Stable | Stable |
| Centrifugation 10K | Stable | Stable | Non |
| | | | Stable |

Comments; Three different complex emulgators were investigated. All (shown above) supported good stable foam. Of the three successful combinations steareth 2 and steareth 21 again and also ceteth 20 and span 80 proved the most powerful emulgator emulsifiers with both compositions showing emulsion stability in the face of 10K centrifugation for 10 mins.

### Example C5

### 49% and 31% Petrolatum with Sucrose Stearic Acid Esters D-1807 (but Without Mineral Oil or an Emollient Like Foam Adjuvant and without ASOS)

| | TLF033 | TLF035 |
|---|---|---|
| White Petrolatum (sofmetic) | 49.00 | 31.00 |
| Sucrose stearic acid esters | 3.00 | 7.00 |
| (mono, di and tri) Surfhope D- | | |
| 1807 | | |
| Water, purified | 48.00 | 62.00 |
| Total | 100.00 | 100.00 |
| Propellant type | 10% 1681 | 10% 1681 |
| Quality | G-E | G-E |
| Color | White | White |
| Odor | No odor | No odor |
| Shakability | Good | Good |
| Centrifugation 1K | 50% | stable |
| | Cream | |

Comment: By increasing the amount of Sucrose stearic acid esters albeit with reduced petroleum it was possible to achieve good quality stable foam. One factor in achieving good stable foam containing a surfactant, comprising a mixture of esters is that the surfactant's HLB is close to RHLB of whole oily phase. Thus, in an embodiment, the surfactant HLB is within about 2 units of the required HLB the whole oil phase and in a preferred embodiment within about 1 unit thereof, particularly when there is a sole surfactant or a sole surfactant and a foam adjuvant or co surfactant.

### Example C6

### 25% and 30% Petrolatum with Liquid Wax and an Emollient Like Foam Adjuvant with and without a Polymeric Agent (but without Mineral Oil or an and without ASOS)

| | TLF034 | TLF036 | TLF037 |
|---|---|---|---|
| White Petrolatum | 25.00 | 25.00 | 30.00 |
| (sofmetic) | | | |
| Cetearyl alcohol | 2.00 | 2.00 | 2.00 |
| Isostearic acid | 25.00 | 25.00 | 30.00 |
| Polysorbate 80 | 3.00 | 3.00 | 3.00 |
| Steareth-2 | 3.00 | 3.00 | 3.00 |
| CMC | | 0.50 | 0.50 |
| Water, purified | 42.00 | 41.50 | 31.50 |
| Total | 100.00 | 100.00 | 100.00 |
| Propellant type | 10% 1681 | 10% 1681 | 10% 1681 |
| Quality | G | G | G |
| Color | White | White | White |
| Odor | No odor | No odor | No odor |
| Shakability | Good | Good | Good |
| | 10% AP- | 10% AP- | 10% AP- |
| | 70 | 70 | 70 |
| | G+ | G+ | G− |
| | White | White | White |
| | No odor | No odor | No odor |
| | Good | Good | Good |

Comment: All the formulations produced good quality foams. Increasing the propellant pressure by using AP70 (a similar hydrocarbon propellant mixture with a substantially higher pressure) appeared to improve slightly the quality of the 25% petrolatum formulations and decreased slightly that of the 30% formulation.

### Example C7

### 49% and 31% Petrolatum with Liquid Wax and with and without an Emollient Like Foam Adjuvant (but without Mineral Oil or ASOS)

| | TLF039 | TLF040 | TLF041 | TLF042 |
|---|---|---|---|---|
| White Petrolatum | 31.00 | 49.00 | 49.00 | 49.00 |
| (sofmetic) | | | | |
| Oleyl alcohol | 15.00 | 15.00 | 15.00 | 15.00 |
| Cetearyl alcohol | 2.00 | 2.00 | | |
| Polysorbate 80 | 3.00 | 3.00 | 3.00 | |
| Steareth-2 | 3.00 | 4.00 | 4.20 | |
| Span 20 (Sorbitan | | | | 7.00 |
| monolaurate) | | | | |
| Water, purified | 46.00 | 27.00 | 28.80 | 29.00 |
| Total | 100.00 | 100.00 | 100.00 | 100.00 |
| Propellant type | 10% 1681 | 10% 1681 | 10% 1681 | 10% 1681 |
| Quality | G-E | G-E | G-E | G-E |
| Color | White | White | White | White |
| Odor | No odor | No odor | No odor | No odor |
| Shakability | Good | Good | Good | Good |
| | 10% AP- | 10% AP- | 10% AP- | 10% AP- |
| | 70 | 70 | 70 | 70 |
| | G-E | G-E | G-E | G-E |
| | White | White | White | White |
| | No odor | No odor | No odor | No odor |
| | Good | Good | Good | Good |

Comment: All the petrolatum/liquid wax formulations produced good quality foam. No significant effect on foam quality was observed after using AP70, a similar hydrocarbon propellant mixture with a substantially higher pressure. The last formulation shows it is possible to achieve good quality foam where a single surfactant is in combination with the liquid wax, which itself can act as a foam booster and contribute to an improved sensation. In the case of the single surfactant having a HLB close to that of the unctuous oil phase appears to be a relevant factor in the success of the foam.

### Example C8

### 42-44% Petrolatum with 18-22% Mineral Oil and a Wax and an Emollient Like Foam Adjuvant

| | TLF048 | TLF049 | TLF050 | TLF048 | TLF051 | TLF052 |
|---|---|---|---|---|---|---|
| White | 44.00 | 42.00 | 42.00 | 44.00 | 42.00 | 44.00 |
| Petrolatum | | | | | | |
| (sofmetic) | | | | | | |
| light Mineral oil | 20.00 | 22.00 | 22.00 | 20.00 | 18.00 | 20.00 |
| Cetearyl alcohol | 2.00 | 1.50 | 2.00 | 2.00 | 2.00 | 2.00 |
| Ceteth-20 | 1.79 | 3.10 | 3.00 | 1.79 | 2.00 | 2.30 |
| (Lipocol C-20) | | | | | | |
| Sorbitan oleate | 2.71 | 1.80 | | 2.71 | | |
| (span 80) | | | | | | |
| Sorbitan laurate | | | | | | 2.00 |
| (span 20) | | | | | | |
| Behenyl alcohol | 0.50 | 0.40 | 1.00 | 0.50 | 0.50 | 0.50 |
| polysorbate 60 | | | | | 1.50 | |
| Water purified | 28.80 | 29.00 | 29.80 | 28.80 | 33.80 | 29.00 |
| Preservative | 0.20 | 0.20 | 0.20 | 0.20 | 0.20 | 0.20 |
| Total | 100.00 | 100.00 | 100.00 | 100.00 | 100.00 | 100.00 |
| Propellant | 10% A- | 8% AP- | 8% AP- | 8% AP- | 8% AP- | 8% AP- |
| | 46 | 70 | 70 | 70 | 70 | 70 |
| Viscosity (cp) | 14316.95 | 12141.41 | 18332.09 | 13709.07 | 10365.79 | |
| Quality | G | G | G | G | G/FG | G+ |
| Color | white | white | white | white | white | white |
| Odor | v.f.o | v.f.o | v.f.o | v.f.o | no odor | no odor |
| Shakability | 0** | 2 | 2 | 1 | 2 | 2 |
| Density | 0.087 | 0.150 | 0.138 | 0.078 | 0.185 | |
| Collapse time | >300/G | >300/ | >300/F | >300 G | >300/ | |
| (sec.) | | FG | | | FG | |
| Hardness (g) | 25.48 | 20.13 | 14.59 | 18.49 | N/R | |
| FTC | | | | | | |
| Quality | G | G | G | G | G+ | |
| Color | white | white | white | White | white | |
| Odor | v.f.o. | v.f.o. | v.f.o. | v.f.o. | no odor | |
| Shakability | 2 | 2 | 2 | 2 | 0** | |

Comment: Small variance in foam quality and other physical properties with relatively small changes in the multi-active agent is observed. In two formulations the shakablity was affected but both formulations remained flowable to enable foam release Interestingly and surprisingly, the example with a single surfactant plus a wax and an emollient like foam adjuvant (all being solids) produced a softer foam even though the pre foam formulation showed a high viscosity. The other examples had a liquid and a solid surfactant. All samples were able to withstand FTC.

### Example C9

### 44-46% Petrolatum with 20% Mineral Oil and an Emollient like Foam Adjuvant

| | TLF053 | TLF054 | TLF055 | TLF056 |
|---|---|---|---|---|
| White Petrolatum | 44.00 | 44.00 | 46.00 | 44.00 |
| (sofmetic) | | | | |
| light Mineral oil | 20.00 | 20.00 | 20.00 | 20.00 |
| Cetearyl alcohol | 2.00 | 2.00 | 2.00 | 2.00 |
| CarboxyMethylCellulose | | | | 0.50 |
| Sodium | | | | |
| Ceteth-10 | 2.30 | 2.10 | 2.20 | 2.30 |
| METHYL GLUCOSE | | 1.10 | 0.70 | |
| SESQUISTEARATE | | | | |
| Sorbitan oleate | 2.00 | 1.00 | 1.50 | 2.00 |
| (span 80) | | | | |
| Behenyl alcohol | 0.50 | 0.50 | 0.50 | 0.50 |
| Water purified | 29.00 | 29.10 | 26.90 | 28.50 |
| Preservative | 0.20 | 0.20 | 0.20 | 0.20 |
| TEA | | | | |
| Total | 100.00 | 100.00 | 100.00 | 100.00 |
| Propellant | 8% AP- | 8% | 8% | 8% AP- |
| | 70 | AP-70 | AP-70 | 70 |
| Centrifugation 1K | phase | | | stable |
| | separation | | | |
| Viscosity (cp) | | | | 10941.67 |
| Quality | G+ | G+ | G+ | G+ |
| Color | white | white | white | white |
| Odor | no odor | no odor | no odor | no odor |
| Shakability | 2 | 2 | 2 | 2 |
| Density | 0.128 | | | 0.115 |
| Collapse time | >300/G | | | >300 |
| (sec.) | | | | |
| Bubble | 105 | 200 | | 167 |
| size(micrometr.) | | | | |
| hardness | | | | 26.10 |

Comment: All the examples provided good quality foam. The addition of a polymeric agent appeared to stabilize the formulation so that it was able to withstand centrifugation. In each case the multi active agent comprised a combination of a liquid and a solid surfactant.

### Example C10

### 44% Petrolatum with 20% Mineral Oil and an Emollient like Foam Adjuvant

| Ingredients | TLF057 |
|---|---|
| White Petrolatum (sofmetic) | 44.00 |
| light Mineral oil | 20.00 |
| Cetearyl alcohol | 2.00 |
| Sorbitan oleate (span 80) | 0.60 |
| Sorbitan laurate (span 20) | 3.20 |
| Laureth-4 | 1.20 |
| Water purified | 28.80 |
| Preservative | 0.20 |
| Total | 100.00 |
| Propellant | 8% AP-70 |
| Centrifugation 1K | 15% |
| | creaming |
| Viscosity (cp) | 12269.38 |
| Quality | G+ |
| Color | white |
| Odor | no odor |
| Shakability | 2 |
| Density | 0.078 |
| Bubble size (micrometr.) | 127 |
| Hardness | 27.53 |
| collapse time (sec.) | >300 |

Comment: it is possible to achieve good stable foam and PFF using a multi active agent comprising only liquid surfactants and Cetearyl alcohol (as foam adjuvant) with, petrolatum and light Mineral oil. The formulation did not separate with centrifugation. All the surfactants had an HLB less than 10 and the two sans had a HLB less than 9.

### Example C11

### About 54% to about 10% Petrolatum with about 10% to about 54% Mineral Oil and a Wax and an Emollient Like Foam Adjuvant

| Ingredients | TLF059 | TLF060 |
|---|---|---|
| White Petrolatum (sofmetic) | 53.30 | 10.70 |
| light Mineral oil | 10.70 | 53.30 |
| Cetearyl alcohol | 2.00 | 2.00 |
| Ceteth-10 | 2.10 | 2.50 |
| Sorbitan oleate (span 80) | 2.20 | |
| Sorbitan laurate (span 20) | | 1.80 |
| Behenyl alcohol | 0.50 | 0.50 |
| Water purified | 29.00 | 29.00 |
| Preservative | 0.20 | 0.20 |
| TEA | | |
| Total | 100.00 | 100.00 |
| Propellant | 8% AP-70 | 8% AP-70 |
| Centrifugation 1K | stable | Stable |
| Viscosity (cp) | 13213.18 | 1714.63 |
| Quality | G+ | G+ |
| Color | white | White |
| Odor | no odor | no odor |
| Shakability | 2 | 2 |
| Density | 0.112 | 0.080 |
| Bubble size (micrometr.) | 160 | 136 |
| hardness | 23.57 | 14.94 |
| collapse time (sec.) | >300 | >300 |

Comment: in order to achieve good stable foam and PFF containing low concentrations of surfactants, while White Petrolatum: light Mineral oil ratio is about 1:5 and about 5:1, RHLB is a factor which should be taken into account. All the surfactants had an HLB less than 13 and the two spans had a HLB less than 9.

### Example C12

### 44-45% Petrolatum with 20-21% Mineral Oil, a Single Agent and with and Without and an Emollient Like Foam Adjuvant

| | TLF068 | TLF069 |
|---|---|---|
| | 11.11.07 | 11.11.08 |
| White Petrolatum | 44.00 | 45.00 |
| (sofmetic) | | |
| light Mineral oil | 20.00 | 21.00 |
| Cetearyl alcohol | 2.00 | |
| Sorbitan laurate | 5.00 | 5.00 |
| (span 20) | | |
| Water purified | 28.80 | 28.80 |
| Preservative | 0.20 | 0.20 |
| Total | 100.00 | 100.00 |
| Propellant | 8% AP-70 | 8% AP-70 |
| Centrifugation 1K | STABLE | SEPARATION |
| Viscosity | 11293.59 | 5998.72 |
| Quality | G+ | G |
| Color | white | white |
| Odor | no odor | no odor |
| Shakability | 2 | 2 |
| Bubble size | 174 | 179 |
| (micrometr.) | | |
| Hardness | 20.70 | 10.79 |
| Collapse time (sec.) | 170 | >300 |

Comment: The multi-active agent comprises a single liquid surfactant and an emollient like foam adjuvant. Good stable foam was achieved in the presence of cetearyl alcohol but its omission resulted in separation upon centrifugation. The surfactant's HLB is close to the RHLB of whole oily phase

What is claimed is:

1. A stable substantially non-alcoholic foamable pharmaceutical emulsion composition comprising:
(1) an unctuous emollient, at a concentration of about 0.5% to about 60% by weight, comprising at least 10% by weight petrolatum;
(2) at least one multi-active agent; at a concentration of about 0.5% to about 15% by weight;
(3) water;
(4) a cosmetic agent or placebo having a degree of solubility of at least one part in 1000 parts in the emulsion composition, in a phase or in an inter-phase thereof; and
(5) at least one liquefied or compressed gas propellant at a concentration of about 3% to about 25% by weight of the total composition so that the ratio of composition other than propellant to propellant is from about 100:3 to about 100:25;
wherein the composition is substantially free of ethanol, propanol, isopropanol, butanol, iso-butanol, t-butanol and pentanol;
wherein the unctuous emollient comprises a petrolatum alone or in combination with other unctuous agents;
wherein the multi active agent is selected from the group consisting of
(a) a non-ionic complex emulgator consisting of two or more emulgators wherein there is a difference of about 4 or more units between the HLB values of two of the emulgators;
(b) a non-ionic surfactant and an additional agent selected from the group consisting of a foam adjuvant, co surfactant, liquid wax and mixtures of any two or more thereof; wherein the surfactant has a HLB within 3 HLB units of the required HLB of the oil phase of the emulsion;
(c) a single non-ionic surfactant without a long polymeric side chain that is composed of a mixture of esters having a HLB close to the required HLB of the oil phase of the emulsion;
(d) combinations of any of the above;
and
wherein the composition can flow, is stored in an pressurized container and upon release expands to form a breakable foam.

2. A composition as claimed in claim 1, wherein upon release from the pressurized container the composition expands to form a breakable foam having a foam hardness in the range of about 5 g to about 50 g, optionally wherein the hardness is in the range of about 8 g to about 40 g, optionally wherein the hardness is in the range of about 10 g to about 30 g, optionally wherein the hardness is in the range of about 11 g to about 20 g; and/or
wherein the unctuous emollient and multi-active agent influence foam hardness such that the foam produced is soft.

3. A composition as claimed in claim 2 wherein the water content is less than 50% by weight of the formulation; and/or
comprising about 1% to about 49% liquid oil, wherein water content is at least about 20% or more of the formulation; optionally wherein the liquid oil is mineral oil.

4. A composition as claimed in claim 2, wherein the surfactant has a HLB within 2 units of the required HLB of the oil phase, optionally wherein the surfactant has a HLB within 1 unit of the required HLB of the oil phase; and/or wherein the multi-active agent comprises a surfactant with a HLB value within about 2 units or within about 1 unit of the required HLB of petrolatum.

5. A composition as claimed in claim 2, additionally comprising a polar solvent; wherein the polar solvent is selected from the group consisting of:
(1) a polyol;
(2) a diol;
(3) a triol;
(4) a solvent selected from the group consisting of propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol, dibutylene glycol, glycerin and 1,2,6-Hexanetriol; and
(5) a combination of any two or more of (1) to (4) above.

6. A composition as claimed in claim 2, wherein the multi active agent is between about 1% to about 10% by weight of the composition; wherein the composition further comprises 0.1% to 5% by weight of a therapeutically active foam adjuvant; wherein the multi-active agent comprises two or more surfactants with a mean HLB below about 9; and/or
wherein the multi-active agent comprises at least one surfactant and at least one additional agent selected from the group consisting of a foam adjuvant, co-surfactant, liquid wax and mixtures of any two or more thereof; wherein the surfactant has a HLB below about 9; and/or
wherein the multi-active agent includes one or more from:
a. combinations of polyoxyethylene alkyl ethers, including ceteth-20 and steareth 10 (Brij 58/Brij 10); ceteth-2 and steareth 10 (Brij 52)/Brij 10); steareth 2 and steareth 20; steareth 2 and steareth 21 (Brij 72/Brij 721); and/or PEG-40 stearate and PEG-100 stearate (Myrj 52/Myrj 59); and/or
b. combinations of sucrose esters, including sucrose stearate (Surfhope 1816)/sucrose distearate (Surfhope 1807); and/or
c. combinations of sorbitan esters, including sorbitan laurate (Span 20)/sorbitan oleate (Span 80); and/or sorbitan laurate (Span 20)/sorbitan stearate (Span 60); and/or
d. combinations of sucrose esters and sorbitan esters, including sucrose stearate (Surphope 1811) and sorbitan stearate (Span 60); and/or
e. combinations of liquid polysorbate detergents and PEG compounds, including polysorbate 80 (Tween 80)/PEG-40 stearate/methyl glucose sequistearate; ceteth-20 and sorbitan oleate (Span 80); polysorbate 80 and Sorbitan oleate (Span 80); polysorbate 80 and steareth 2; sorbitan oleate (Span 80), sorbitan laurate (Span 20) and laureth-4; ceteth 20 and polysorbate 60; and or sorbitan oleate and polysorbate 60; and/or
f. a foam adjuvant or cosurfactant and any of the following: sorbitan laurate (span 20); sorbitan monopalmitate (span 40); sorbitan stearate (span 60); sorbitan oleate (span 80); ceteth-20; acrylates/C10-30 Alkyl acrylate crosspolymer (Pemulen TR1 or TR2); sorbitan stearate and sucrose cocoate (Arlatone 2121), sodium stearyl phthalamate (Stepan Mild RM1), Polyglyceryl-10 pentastearate and behenyl alcohol and sodium stearoyl lactylate (Nikomulese 41); cetearyl alcohol and cetearyl glucoside (Montanov 68); an or including ceteth 20 and behenyl alcohol; and/or
g. a foam adjuvant and emulgators, wherein the emulgators include ceteth-20, sorbitan oleate (span 80); ceteth 20 and polysorbate 60; ceteth-10, sorbitan oleate (span 80); and or ceteth-10, sorbitan laurate (span 20).; and/or
h. a foam adjuvant and a polymeric agent including methocel and xanthan gum or carboxymethylcellulose sodium;
i. sucrose stearic acid esters; sorbitan fatty acid esters with or without cetearyl alcohol; and/or
j. Sorbitan laurate (span 20); or sorbitan monopalmitate (span 40) and a liquid wax including sorbitan laurate (span 20); or sorbitan monopalmitate (span 40) and/or isostearic acid or oleyl alcohol; and/or
k. combinations of sucrose stearate and sorbitan stearate (and) sucrose cocoate (arlacel); glyceryl monostearate and/or ceteth 10; and/or cetearyl glucoside and sorbitan stearate; optionally wherein the surfactant is selected from the group comprising sorbitan laurate (span 20), sorbitan monopalmitate (span 40), sorbitan stearate (span 60) and/or sorbitan oleate (span 80).

7. A composition as claimed in claim 2, wherein the cosmetic agent or placebo is insoluble or very slightly soluble in water or in the unctuous emollient and the composition is formulated so that
a) the resultant foam when applied topically to a target will nor per se form an effective occlusive barrier, is not completely occlusive; or is not sufficient to form an occlusive barrier or any occlusiveness is significantly transient; or
b) the composition does not comprise an organic co solvent.

8. A composition as claimed in claim 2, wherein the composition is non-flammable, wherein said liquefied or compressed gas propellant contains hydrofluorocarbon.

9. A composition as claimed in claim 2, further comprising liquid wax; wherein water is at least about 20% or more of the formulation; and wherein the ratio between petrolatum and the liquid wax ranges between about 1:4 to about 10:1, optionally wherein the liquid wax includes one or more of isostearic acid, caprylic acid, capric acid, oleyl alcohol, isostearic alcohol, capric alcohol, capryl alcohol and/or jojoba oil.

10. A composition as claimed in claim 2, wherein the unctuous emollient comprises a combination of petrolatum and oil and wherein the ratio of oil to petrolatum is selected from the group consisting of a) ranging between about 1:6 and about 6:1; b) ranging between about 1:4 to and about 2:1; c) ranging between about 1:3 and about 1:1; and d) ranging between about 1:3 and about 1:2; or
wherein the unctuous emollient comprises a combination of petrolatum, an oil and an emollient foam adjuvant and wherein the ratio between the unctuous emollient and the emulsifiying agent (excluding foam adjuvants/co-surfactants) is, in excess of 8:1; or
wherein the unctuous emollient is a combination of petrolatum and an emollient foam adjuvant and wherein the ratio between petrolatum and the foam adjuvant ranges is selected from the group consisting of a) ranging between about 70:1 to about 2:1; and ranging between about 30:1 and about 8:1.

11. A composition as claimed in claim 1, further comprising an ionic surfactant wherein the ratio of non-ionic surfactant or non-ionic emulgators to ionic surfactant is greater than about 6:1 or is in the range of about 100:1 to about 1:1.

12. A method of treating, alleviating or preventing a dermatological or mucosal disorder, comprising: applying a composition onto a mammalian body skin or mucosal surface having a dermatological or mucosal disorder in need of treatment, or is susceptible to a dermatological or mucosal disorder said composition comprising the composition of claim 1.

13. A stable substantially non-alcoholic foamable pharmaceutical emulsion composition comprising:
(1) a petrolatum at a concentration of about 10% to about 30% by weight;
(2) a non ionic surface active agent at concentration of about 0.5% to about 15% by weight;
(3) water;
(4) a cosmetic agent or placebo having a degree of solubility of at least one part in 1000 parts in the emulsion composition or a phase or inter-phase thereof selected from the group consisting of glycerin, a silicone or mixtures thereof;
(5) a liquid oil;
(6) a foam adjuvant; and
(7) at least one liquefied or compressed gas propellant at a concentration of about 3% to about 25% by weight of the total composition so that the ratio of composition other than propellant to propellant is from about 100:3 to about 100:25;
wherein the composition is substantially free of ethanol, propanol, isopropanol, butanol, iso-butanol, t-butanol and pentanol;
wherein the composition can flow; and wherein the composition is resistant to separation and or creaming, is stored in a pressurized container and upon release expands to form a breakable foam.

14. The composition of claim 13, wherein the cosmetic agent is glycerin and a silicone.

15. A composition as claimed in claim 1, further comprising a polymeric agent wherein the polymeric agent is 0.01% to 5% by weight and includes at least one of a bioadhesive agent, a gelling agent, a film forming agent and/or a phase change agent.

16. A composition as claimed in claim 14, wherein the composition is resistant to separation and or creaming.

17. The composition as claim in claim 16, wherein the composition is not separate visibly upon examination immediately after the formulation has been exposed to centrifugation at 1000 rpm for about 10 minutes.

18. The composition as claimed in claim 16, wherein the composition is not separate visibly upon examination immediately stable after the formulation has been exposed to centrifugation at 3000 rpm for about 10 minutes.

19. A composition as claimed in claim 14, wherein the composition prior to addition of propellant has a viscosity measured at a spindle speed of 10 rpm is in the range of about 1000 cP to about 30000 cP.

20. A composition as claimed in claim 14, wherein the composition prior to addition of propellant has a viscosity measured at a spindle speed of 10 rpm is in the range of about 5000 cP to about 10000 cP.

21. A composition as claimed in claim 14, wherein the composition underwent two FTC cycles without change in foam appearance.

22. A composition as claimed in claim 14, wherein the composition, wherein the composition upon release from the pressurized container expands to form a breakable foam having a foam hardness of less than about 20 g.

23. A composition as claimed in claim 14, wherein the composition further comprises a preservative.

24. A composition as claimed in claim 14, wherein the non ionic surfactant comprises at least one polyethelyne glycol ether of stearyl alcohol.

25. A composition as claimed in claim 14, wherein the ratio of liquid oil to petrolatum ranges between about 1:3 to about 1:1.

26. A method of treating, alleviating or preventing a dermatological or mucosal disorder, comprising: applying a composition onto a mammalian body skin or mucosal surface having a dermatological or mucosal disorder in need of treatment, or is susceptible to a dermatological or mucosal disorder said composition comprising the composition of claim 13.
